(19) United States
(12) Patent Application Publication
(10) Pub. No.: US 2026/0191985 A1
(43) Pub. Date: Jul. 9, 2026

(54) PTK7 ANTIBODY CONJUGATES

(71) Applicants: WUXI BIOLOGICS (Shanghai FX) Co., Ltd., Shanghai (CN); HANGZHOU DAC BIOTECHNOLOGY CO., LTD., Zhejiang (CN)

(72) Inventors: Xia WANG, Shanghai (CN); Robert Yongxin ZHAO, Zhejiang (CN); Qingling LU, Shanghai (CN); Qingliang YANG, Zhejiang (CN); Lei WU, Shanghai (CN); Yuanyuan HUANG, Zhejiang (CN); Jijie GU, Shanghai (CN); Hangbo YE, Zhejiang (CN); Juan WANG, Zhejiang (CN)

(21) Appl. No.: 19/461,052

(22) Filed: Jan. 27, 2026

(30) Foreign Application Priority Data
Jan. 9, 2025 (WO) PCT/CN2025/071570
Dec. 9, 2025 (WO) PCT/CN2025/141205
Jan. 8, 2026 (WO) PCT/CN2026/071399

(51) Int. Cl. A61K 47/68

(52) U.S. Cl. CPC A61K 47/6871; A61K 47/68037; A61K 47/6889

(57) ABSTRACT

The present disclosure generally relates to conjugates, compounds, and compositions comprising a PTK7 antibody, and to methods of using the conjugates for treating cancers and tumors.

### CROSS-REFERENCE

This application claims the benefit of PCT/CN2025/071570, filed on Jan. 9, 2025, PCT/CN2025/141205, filed Dec. 9, 2025, and PCT/CN2026/071399, filed Jan. 8, 2026, all of which the entirety of which is hereby incorporated by reference herein.

### SEQUENCE LISTING

The instant application contains a Sequence Listing which has been submitted electronically in XML file format and is hereby incorporated by reference in its entirety. Said XML copy, created on Jan. 12, 2026, is named 45455-704_603_generic_SL.xml and is 117,681 bytes in size.

### BACKGROUND

Antibody or antibody drug conjugates (ADC) have been utilized for treatment of diseases or conditions (e.g., solid tumor or cancer). However, current antibodies or ADCs have exhibited limitations such as lack of therapeutic efficacy or off-target toxicity.

### SUMMARY

Accordingly, there remains a need for an antibody or ADC to effectively target and/or deliver a drug for treating a disease or condition described herein.

Provided herein are conjugates of Formula (I): X-[L-D]ₙ, wherein: n is a number from 1 to 20; X is a polypeptide that binds PTK7 and comprises a heavy chain complementarity determining region 3 (HCDR3) comprising an amino acid sequence that has at least 85% homology to SEQ ID NO: 7; L is a linker that covalently links X to D; D is a drug, and L-D has a structure of Formula (II):

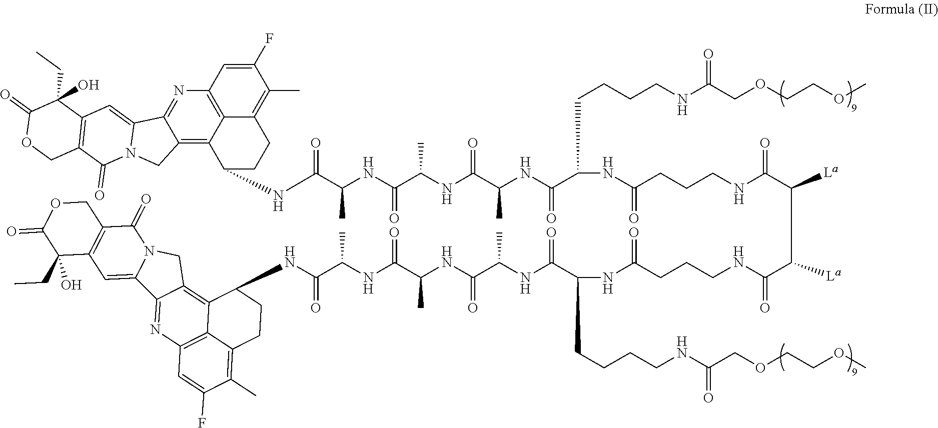

or a stereoisomer thereof, or a pharmaceutically acceptable salt thereof, wherein each L^{a} is independently

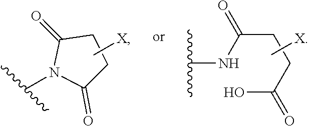

Further provided herein are conjugates wherein the polypeptide comprises an amino acid sequence with at least 75%, at least 80%, at least 85%, at least 90%, at least 95%, or at least 99% homology to SEQ ID NO: 1. Further provided herein are conjugates wherein the polypeptide comprises an amino acid sequence with at least 75%, at least 80%, at least 85%, at least 90%, at least 95%, or at least 99% homology to SEQ ID NO: 2. Further provided herein are conjugates wherein the polypeptide comprises first amino acid sequence with at least 75%, at least 80%, at least 85%, at least 90%, at least 95%, or at least 99% homology to SEQ ID NO: 1; and a second amino acid sequence with at least 75%, at least 80%, at least 85%, at least 90%, at least 95%, or at least 99% homology to SEQ ID NO: 2. Further provided herein are conjugates wherein the polypeptide comprises a first amino acid sequence that is SEQ ID NO: 1; and a second amino acid sequence that is SEQ ID NO: 2. Further provided herein are conjugates wherein the polypeptide comprises an amino acid sequence that is at least 50 contiguous amino, at least contiguous 100 amino acids, at least 150 contiguous amino, or at least contiguous 200 amino acids homology to SEQ ID NO: 1. Further provided herein are conjugates wherein the polypeptide comprises an amino acid sequence that is at least 50 contiguous amino, at least contiguous 100 amino acids, at least 150 contiguous amino, or at least contiguous 200 amino acids homology to SEQ ID NO: 2. Further provided herein are conjugates wherein the polypeptide comprises a first amino acid sequence that is at least 50 contiguous amino, at least contiguous 100 amino acids, at least 150 contiguous amino, or at least contiguous 200 amino acids homology to SEQ ID NO: 1; and a second amino acid sequence that is at least 50 contiguous amino, at least contiguous 100 amino acids, at least 150 contiguous amino, or at least contiguous 200 amino acids homology to SEQ ID NO: 2. Further provided herein are conjugates wherein the polypeptide comprises an amino acid sequence with at least 75%, at least 80%, at least 85%, at least 90%, at least 95%, or at least 99% homology to SEQ ID NO: 3. Further provided herein are conjugates wherein the polypeptide comprises an amino acid sequence with at least 75%, at least 80%, at least 85%, at least 90%, at least 95%, or at least 99% homology to SEQ ID NO: 4. Further provided herein are conjugates wherein the polypeptide comprises a first amino acid sequence with at least 75%, at least 80%, at least 85%, at least 90%, at least 95%, or at least 99% homology to SEQ ID NO: 3; and a second amino acid sequence with at least 75%, at least 80%, at least 85%, at least 90%, at least 95%, or at least 99% homology to SEQ ID NO: 4. Further provided herein are conjugates wherein, wherein the polypeptide comprises a first amino acid sequence that is SEQ ID NO: 3; and a second amino acid sequence that is SEQ ID NO: 4. Further provided herein are conjugates wherein the polypeptide comprises an amino acid sequence that is at least 50 contiguous amino acids or at least contiguous 100 amino acids homology to SEQ ID NO: 3. Further provided herein are conjugates wherein the polypeptide comprises an amino acid sequence that is at least 50 contiguous amino acids or at least contiguous 100 amino acids homology to SEQ ID NO: 4. Further provided herein are conjugates wherein the polypeptide comprises a heavy chain complementarity determining region 1 (HCDR1) comprising an amino acid sequence with at least 75%, at least 80%, at least 85%, at least 90%, at least 95%, or at least 99% homology to SEQ ID NO: 5. Further provided herein are conjugates wherein the polypeptide comprises a heavy chain complementarity determining region 2 (HCDR2) comprising an amino acid sequence with at least 75%, at least 80%, at least 85%, at least 90%, at least 95%, or at least 99% Further provided herein are conjugates wherein the polypeptide comprises a heavy chain complementarity determining region 3 (HCDR3) comprising an amino acid sequence that is at least 90%, at least 95%, or at least 99% homology to SEQ ID NO: 7. Further provided herein are conjugates wherein the polypeptide comprises a light chain complementarity determining region 1 (LCDR1) comprising an amino acid sequence with at least 75%, at least 80%, at least 85%, at least 90%, at least 95%, or at least 99% homology to SEQ ID NO: 8. Further provided herein are conjugates wherein the polypeptide comprises a light chain complementarity determining region 2 (LCDR2) comprising an amino acid sequence with at least 75%, at least 80%, at least 85%, at least 90%, at least 95%, or at least 99% homology to SEQ ID NO: 9. Further provided herein are conjugates wherein the polypeptide comprises a light chain complementarity determining region 3 (LCDR3) comprising an amino acid with at least 75%, at least 80%, at least 85%, at least 90%, at least 95%, or at least 99% homology to SEQ ID NO: 10. Further provided herein are conjugates wherein the polypeptide comprises a HCDR1 comprising an amino acid sequence that is SEQ ID NO: 5; a HCDR2 comprising an amino acid sequence that is SEQ ID NO: 6; a HCDR3 comprising an amino acid sequence that is SEQ ID NO: 7; a LCDR1 comprising an amino acid sequence that is SEQ ID NO: 8; a LCDR2 comprising an amino acid sequence that is SEQ ID NO: 9; and a LCDR3 comprising an amino acid sequence that is SEQ ID NO: 10. Further provided herein are conjugates wherein the polypeptide binds to an epitope comprising an amino acid sequence with at least 75%, at least 80%, at least 85%, at least 90%, at least 95%, or at least 99% homology to SEQ ID NO: 11. Further provided herein are conjugates wherein the polypeptide binds to PTK7. Further provided herein are conjugates wherein the polypeptide is chimeric or humanized. Further provided herein are conjugates wherein the polypeptide comprises a Fc region. Further provided herein are conjugates wherein, wherein the Fc region comprises a human IgG1 Fc region. Further provided herein are conjugates wherein, wherein the Fc region comprises at least one modification. Further provided herein are conjugates wherein, wherein the at least one modification comprises a L234A (Kabat numbering) mutation or a L235A (Kabat numbering) mutation. Further provided herein are conjugates wherein, wherein the at least one modification comprises the L234A (Kabat numbering) mutation and the L235A (Kabat numbering) mutation. Further provided herein are conjugates comprising one or more sequences of Tables 2-4. Further provided herein are conjugates comprising one or more sequences of Tables 3-4.

Provided herein are pharmaceutical compositions comprising the conjugate provided herein and a pharmaceutically acceptable diluent, carrier, or excipient. Further provided herein are pharmaceutical compositions wherein the pharmaceutical composition is formulated for administering intratumorally, intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, or a combination thereof. Further provided herein are pharmaceutical compositions wherein the pharmaceutical composition is for treating a disease or condition. Further provided herein are pharmaceutical compositions wherein the disease or condition comprises solid tumor. Further provided herein are pharmaceutical compositions wherein the disease or condition comprises cancer. Further provided herein are pharmaceutical compositions wherein the cancer comprises non-small cell lung cancer (NSCLC); ovarian cancer; breast cancer; gastrointestinal cancer; prostate cancer; head and neck cancer; endometrial cancer; cervical cancer; or a combination thereof. Further provided herein are pharmaceutical compositions comprising one or more sequences of Tables 2-4. Further provided herein are pharmaceutical compositions comprising one or more sequences of Tables 3-4.

Provided herein are methods of treating a disease or condition in subject, comprising: administering the subject with a conjugate or pharmaceutical composition provided herein, thereby treating the disease or condition in the subject.

Provided herein are methods of delivering a drug to a cell in the subject, comprising: administering the subject with a conjugate or pharmaceutical composition provided herein.

Provided herein are methods of delivering a drug to a subject, comprising: administering the subject a conjugate of Formula (I): X-[L-D]ₙ, wherein: n is a number from 1 to 20; X is a polypeptide comprising a heavy chain complementarity determining region 3 (HCDR3) comprising an amino acid sequence that has at least 85% homology to SEQ ID NO: 7; L is a linker that covalently links X to D; D is a drug, and L-D has a structure of Formula (II):

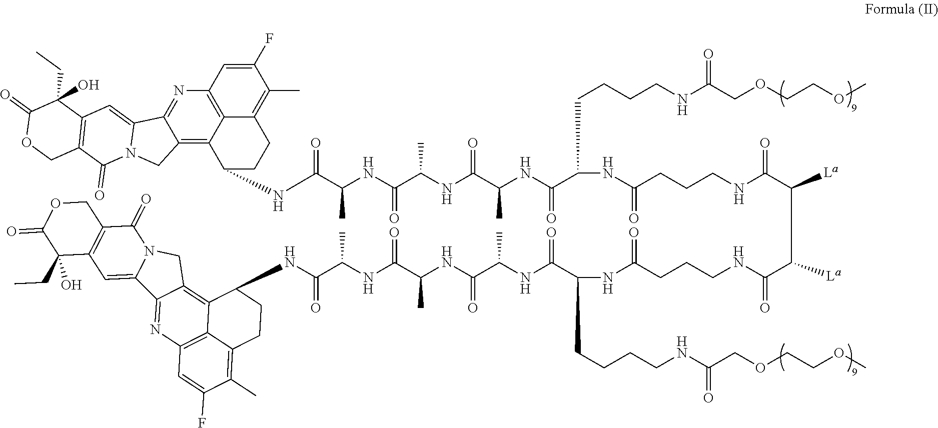

or a stereoisomer thereof, or a pharmaceutically acceptable salt thereof, wherein each L^{a} is independently

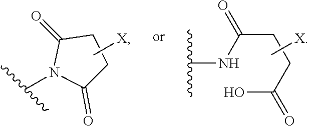

Provided herein are methods of treating a disease or condition in a subject, comprising: contacting a cell of the subject with a conjugate of Formula (I): X-[L-D]ₙ, wherein: n is a number from 1 to 20; X is a polypeptide comprising a heavy chain complementarity determining region 3 (HCDR3) comprising an amino acid sequence that has at least 85% homology to SEQ ID NO: 7; L is a linker that covalently links X to D; D is a drug, and L-D has a structure of Formula (II):

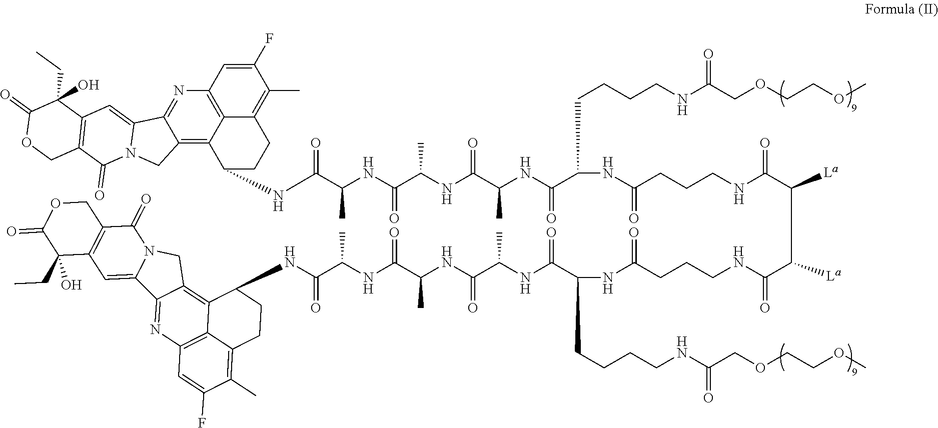

or a stereoisomer thereof, or a pharmaceutically acceptable salt thereof, wherein each L^{a} is independently

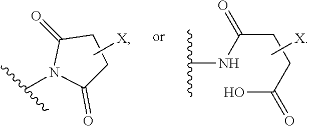

Further provided herein are methods wherein the polypeptide comprises an amino acid sequence with at least 75%, at least 80%, at least 85%, at least 90%, at least 95%, or at least 99% homology to SEQ ID NO: 1. Further provided herein are methods wherein the polypeptide comprises an amino acid sequence with at least 75%, at least 80%, at least 85%, at least 90%, at least 95%, or at least 99% homology to SEQ ID NO: 2. Further provided herein are methods wherein the polypeptide comprises a first amino acid sequence with at least 75%, at least 80%, at least 85%, at least 90%, at least 95%, or at least 99% homology to SEQ ID NO: 1; and a second amino acid sequence with at least 75%, at least 80%, at least 85%, at least 90%, at least 95%, or at least 99% homology to SEQ ID NO: 2. Further provided herein are methods wherein the polypeptide comprises a first amino acid sequence that is SEQ ID NO: 1; and a second amino acid sequence that is SEQ ID NO: 2. Further provided herein are methods wherein the polypeptide comprises an amino acid sequence that is at least 50 contiguous amino, at least contiguous 100 amino acids, at least 150 contiguous amino, or at least contiguous 200 amino acids homology to SEQ ID NO: 1. Further provided herein are methods wherein the polypeptide comprises an amino acid sequence that is at least 50 contiguous amino, at least contiguous 100 amino acids, at least 150 contiguous amino, or at least contiguous 200 amino acids homology to SEQ ID NO: 2. Further provided herein are methods wherein the polypeptide comprises a first amino acid sequence that is at least 50 contiguous amino, at least contiguous 100 amino acids, at least 150 contiguous amino, or at least contiguous 200 amino acids homology to SEQ ID NO: 1; and a second amino acid sequence that is at least 50 contiguous amino, at least contiguous 100 amino acids, at least 150 contiguous amino, or at least contiguous 200 amino acids homology to SEQ ID NO: 2. Further provided herein are methods wherein the polypeptide comprises an amino acid sequence with at least 75%, at least 80%, at least 85%, at least 90%, at least 95%, or at least 99% homology to SEQ ID NO: 3. Further provided herein are methods wherein the polypeptide comprises an amino acid sequence with at least 75%, at least 80%, at least 85%, at least 90%, at least 95%, or at least 99% homology to SEQ ID NO: 4. Further provided herein are methods wherein the polypeptide comprises a first amino acid sequence with at least 75%, at least 80%, at least 85%, at least 90%, at least 95%, or at least 99% homology to SEQ ID NO: 3; and a second amino acid sequence with at least 75%, at least 80%, at least 85%, at least 90%, at least 95%, or at least 99% homology to SEQ ID NO: 4. Further provided herein are methods wherein the polypeptide comprises a first amino acid sequence that is SEQ ID NO: 3; and a second amino acid sequence that is SEQ ID NO: 4. Further provided herein are methods wherein the polypeptide comprises an amino acid sequence that is at least 50 contiguous amino acids or at least contiguous 100 amino acids homology to SEQ ID NO: 3. Further provided herein are methods wherein the polypeptide comprises an amino acid sequence that is at least 50 contiguous amino acids or at least contiguous 100 amino acids homology to SEQ ID NO: 4. Further provided herein are methods wherein the polypeptide comprises a heavy chain complementarity determining region 1 (HCDR1) comprising an amino acid sequence with at least 75%, at least 80%, at least 85%, at least 90%, at least 95%, or at least 99% homology to SEQ ID NO: 5. Further provided herein are methods wherein the polypeptide comprises a heavy chain complementarity determining region 2 (HCDR2) comprising an amino acid sequence with at least 75%, at least 80%, at least 85%, at least 90%, at least 95%, or at least 99% homology to SEQ ID NO: 6. Further provided herein are methods wherein the polypeptide comprises a heavy chain complementarity determining region 3 (HCDR3) comprising an amino acid sequence that is at least 90%, at least 95%, or at least 99% homology to SEQ ID NO: 7. Further provided herein are methods wherein the polypeptide comprises a light chain complementarity determining region 1 (LCDR1) comprising an amino acid sequence with at least 75%, at least 80%, at least 85%, at least 90%, at least 95%, or at least 99% homology to SEQ ID NO: 8. Further provided herein are methods wherein the polypeptide comprises a light chain complementarity determining region 2 (LCDR2) comprising an amino acid sequence with at least 75%, at least 80%, at least 85%, at least 90%, at least 95%, or at least 99% homology to SEQ ID NO: 9. Further provided herein are methods wherein the polypeptide comprises a light chain complementarity determining region 3 (LCDR3) comprising an amino acid with at least 75%, at least 80%, at least 85%, at least 90%, at least 95%, or at least 99% homology to SEQ ID NO: 10. Further provided herein are methods wherein the polypeptide comprises a HCDR1 comprising an amino acid sequence that is SEQ ID NO: 5; a HCDR2 comprising an amino acid sequence that is SEQ ID NO: 6; a HCDR3 comprising an amino acid sequence that is SEQ ID NO: 7; a LCDR1 comprising an amino acid sequence that is SEQ ID NO: 8; a LCDR2 comprising an amino acid sequence that is SEQ ID NO: 9; and a LCDR3 comprising an amino acid sequence that is SEQ ID NO: 10. Further provided herein are methods wherein the polypeptide binds to an epitope comprising an amino acid sequence with at least 75%, at least 80%, at least 85%, at least 90%, at least 95%, or at least 99% homology to SEQ ID NO: 11. Further provided herein are methods wherein the polypeptide binds to PTK7. Further provided herein are methods wherein the polypeptide is chimeric or humanized. Further provided herein are methods wherein the polypeptide comprises a Fc region. Further provided herein are methods wherein the Fc region comprises a human IgG1 Fc region. Further provided herein are methods wherein the Fc region comprises at least one modification. Further provided herein are methods wherein the at least one modification comprises a L234A (Kabat numbering) mutation or a L235A (Kabat numbering) mutation. Further provided herein are methods wherein the at least one modification comprises the L234A (Kabat numbering) mutation and the L235A (Kabat numbering) mutation. Further provided herein are methods wherein the disease or condition comprises solid tumor. Further provided herein are methods wherein the disease or condition comprises cancer. Further provided herein are methods wherein the cancer comprises non-small cell lung cancer (NSCLC); ovarian cancer; breast cancer; gastrointestinal cancer; prostate cancer; head and neck cancer; endometrial cancer; cervical cancer; or a combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A illustrates comparable antigen (PTK7) binding exhibited by the PTK7 antibody and the PTK7 antibody conjugate (ADC-A) to NCI-H520 cells. The x-axis is labeled CONC (nM) from 10⁻³ to 10² on a base10 logarithmic scale; the y-axis is labeled MFI from 0 to 8000 at 2000 unit intervals.

FIG. 1B illustrates comparable internalization exhibited by the PTK7 antibody and the ADC-A. The x-axis is labeled CONC (nM) from 10⁻³ to 10² on a base10 logarithmic scale; the y-axis is labeled Spots per cell from 0 to 20 at 5 unit intervals. PTK7 antibody ADC-A targeted PTK7 Ig domain, where the PTK7 antibody was IgG1 class antibody and exhibited by a binding affinity (Kd) of 0.16 nM). The conjugate comprised a linker comprising a proteolytically cleavable tri-alanine. The conjugate comprised a linker-payload 1A, where the payload was a topoisomerase 1 inhibitor. The drug to antibody ratio (DAR) of the conjugate tested was six. Key: Isotype-1A (shaded triangle); Isotype (shaded circle); ADC-A (dark shaded square); PTK-7 mAb (light shaded square).

FIGS. 2A-2E illustrate improved therapeutic efficacy of ADC-A described herein compared to cofetuzumab (Cofe-P) and an isotype-1A ADC (control ADC without antigen binding property). FIG. 2A illustrates that ADC-A (inverted triangles) increased in vitro cytotoxicity in NCI-H146 lung carcinoma cells compared to cofetuzumab (Cofe-P, squares) or the isotype-1A ADC (circles). The x-axis is labeled concentration from 10⁻⁴ to 10² on a base10 logarithmic scale; the y-axis is labeled cytotoxicity (%) from −20 to 100 at 20 unit intervals. FIG. 2B illustrates that ADC-A (inverted triangles) increased dose-dependent in vitro cytotoxicity in OVCAR-3 adenocarcinoma of the ovary cells compared to cofetuzumab (Cofe-P, squares) or isotype-1A ADC control (circles). The x-axis is labeled concentration from 10⁻⁴ to 10² on a base10 logarithmic scale; the y-axis is labeled cytotoxicity (%) from −20 to 100 at 20 unit intervals.

FIG. 2C illustrates therapeutic efficacy of tumor reduction of cell line-derived NCI-H446 xenograft (CDX) model of a conjugate described herein (ADC-A, triangles) compared to tumor induced by a comparable antibody conjugate (cofetuzumab, “Cofe-P”, squares) and controls (circles). X-axis referenced days post injection with arrows indicating days of injection. Key: PBS (shaded circles); Isotype-1A ADC (Isotype-CPT-113, 4 mg/kg, half shaded circles); Free drug (CPT-116, 0.078 mg/kg, unshaded circles); Cofe-P (4 mg/kg, shaded squares); Cofe-P (1 mg/kg, half shaded squares); Cofe-P (0.25 mg/kg, unshaded squares); ADC-A (4 mg/kg, shaded triangles); ADC-A (1 mg/kg, half shaded triangles); ADC-A (0.25 mg/kg, unshaded triangles). The x-axis is labeled days after first treatment from 0 to 35 at 7 day intervals; the y-axis is labeled tumor volume (mm³) from 0 to 3000 at 1000 mm³ intervals.

FIG. 2D illustrates body weights of the mice of the NCI-H446 xenograft (CDX) model treated in FIG. 2C.

FIG. 2E illustrates that ADC-A (triangles) increased tumor growth inhibition in OVCAR-3 xenograft model to cofetuzumab (Cofe-P, squares). Key: PBS (shaded circles); Cofe-P (0.6 mg/kg, unshaded squares); Cofe-P (3 mg/kg, shaded squares); ADC-A (0.6 mg/kg, unshaded triangles); ADC-A (3 mg/kg, shaded triangles). The x-axis is labeled days after treatment from 0 to 42 at 7 day intervals; the y-axis is labeled tumor volume (mm³) from 0 to 2000 at 400 mm³ intervals.

FIG. 2F illustrates therapeutic efficacy against OVCAR3 ovarian cancer cell line-derived xenograft model.

FIG. 3A illustrates therapeutic efficacy of tumor reduction of cell line-derived HCC70 triple-negative breast cancer (TNBC) xenograft (CDX) model of a conjugate described herein (ADC-A, squares) compared to tumor induced by a comparable antibody conjugate (cofetuzumab, “Cofe-P”, circles) and controls (diamonds). X-axis referenced days post injection with arrows indicating days of injection. Key: DPBS (solid diamonds); Isotype-1A ADC (Isotype-CPT-113, 3 mg/kg, half shaded diamonds); Free drug (CPT-116, 0.058 mg/kg, unshaded diamonds); Cofe-P (3 mg/kg, solid circles); Cofe-P (1 mg/kg, half shaded circles); Cofe-P (0.3 mg/kg, unshaded circles); ADC-A (3 mg/kg, solid squares); ADC-A (1 mg/kg, half shaded squares); ADC-A (0.3 mg/kg, unshaded squares). The x-axis is labeled days after first treatment from 0 to 35 at 7 day intervals; the y-axis is labeled tumor volume (mm³) from 0 to 2500 at 500 mm³ intervals.

FIG. 3B illustrates body weights of the mice of the HCC70 TNBC xenograft (CDX) model treated in FIG. 3A.

FIG. 4A illustrates therapeutic efficacy of tumor reduction of cell line-derived NCI-H146 small cell lung carcinoma (SCLC) xenograft (CDX) model of a conjugate described herein (ADC-A, squares) compared to tumor induced by a comparable antibody conjugate (cofetuzumab, “Cofe-P”, circles) and controls (diamonds). X-axis referenced days post injection with arrows indicating day of injection. Key: DPBS (solid diamonds); Isotype-1A ADC (Isotype-CPT-113, 3 mg/kg, half shaded diamonds); Cofe-P (3 mg/kg, solid circles); Cofe-P (1 mg/kg, half shaded circles); Cofe-P (0.3 mg/kg, unshaded circles); ADC-A (3 mg/kg, solid squares); ADC-A (1 mg/kg, half shaded squares); ADC-A (0.3 mg/kg, unshaded squares). The x-axis is labeled days after first treatment from 0 to 35 at 7 day intervals; the y-axis is labeled tumor volume (mm³) from 0 to 2500 at 500 mm³ intervals.

FIG. 4B illustrates body weights of the mice of the NCI-H146 SCLC xenograft (CDX) model treated in FIG. 4A.

FIG. 5A illustrates therapeutic efficacy of tumor reduction of cell line-derived NCI-H69 SCLC cell line-derived xenograft (CDX) model of a conjugate described herein (ADC-A, squares) compared to tumor induced by a comparable antibody conjugate (cofetuzumab, “Cofe-P”, circles) and controls (diamonds). X-axis referenced days post injection with arrows indicating day of injection. Key: DPBS (solid diamonds); Isotype-1A ADC (Isotype-CPT-113, 3 mg/kg, half shaded diamonds); Cofe-P (3 mg/kg, solid circles); Cofe-P (0.6 mg/kg, half shaded circles); ADC-A (3 mg/kg, solid squares); and ADC-A (0.6 mg/kg, unshaded squares). The x-axis is labeled days after first treatment from 0 to 35 at 7 day intervals; the y-axis is labeled tumor volume (mm³) from 0 to 1000 at 200 mm³ intervals.

FIG. 5B illustrates body weights of the mice of the NCI-H69 SCLC xenograft (CDX) model treated in FIG. 5A.

FIG. 6A illustrates therapeutic efficacy of tumor reduction of cell line-derived HCT-116 colorectal cancer cell (CRC) cell line-derived xenograft (CDX) model of a conjugate described herein (ADC-A, squares) compared to tumor induced by a comparable antibody conjugate (cofetuzumab, “Cofe-P”, circles) and controls (diamonds). X-axis referenced days post injection with arrows indicating day of injection. Key: DPBS (solid diamonds); Isotype-1A ADC (Isotype-CPT-113, 4.5 mg/kg, half shaded diamonds); Cofe-P (4.5 mg/kg, solid circles); Cofe-P (1.5 mg/kg, half shaded circles); Cofe-P (0.5 mg/kg, unshaded circles); ADC-A (4.5 mg/kg, solid squares); ADC-A (1.5 mg/kg, half shaded circles); and ADC-A (0.5 mg/kg, unshaded squares). The x-axis is labeled days after first treatment from 0 to 35 at 7 day intervals; the y-axis is labeled tumor volume (mm³) from 0 to 2500 at 500 mm³ intervals.

FIG. 6B illustrates body weights of the mice of the HCT-116 CRC xenograft (CDX) model treated in FIG. 6A.

### DETAILED DESCRIPTION

This disclosure is directed, at least in part, to the field of drugs and drug conjugates, specifically conjugates of cell proliferation inhibitor drugs and a polypeptide (including, for example, an antibody or antigen-binding fragment). In some embodiments, the polypeptide (e.g., an antibody or antigen-binding fragment) binds to an epitope of protein tyrosine kinase 7 (PTK7). PTK7 expression has been linked to cancer growth and migration in endometrial cancer, prostate cancer, cervical cancer, head and neck cancer, breast cancer, colorectal cancer, esophageal cancer, lung cancer, ovarian cancer, or gastric cancer. Current therapy targeting PTK7 expression has limitation such as lack of therapeutic efficacy or off-target toxicity. Accordingly, described herein are conjugates (e.g., a polypeptide for targeting PTK7 conjugated to a linker and payload) and methods for targeting and delivering a drug to a site of PTK7 expression. For example, the conjugate can deliver the drug (e.g., a topoisomerase described herein) to a cancer cell or a tumor environment expressing PTK7.

In some embodiments, the conjugate comprises a structure of Formula (I): X-[L-D]ₙ, where n is a number from 1 to 20. In some embodiments, n is a number of about 6. In some embodiments, X is a polypeptide comprising a heavy chain complementarity determining region 3 (HCDR3) comprising an amino acid sequence that has at least 85% homology to SEQ ID NO: 7. In some embodiments, L is a linker that covalently links X to D. In some embodiments, D is a drug such as a topoisomerase or an analog described herein. In some embodiments, the L-D has a structure of Formula (II):

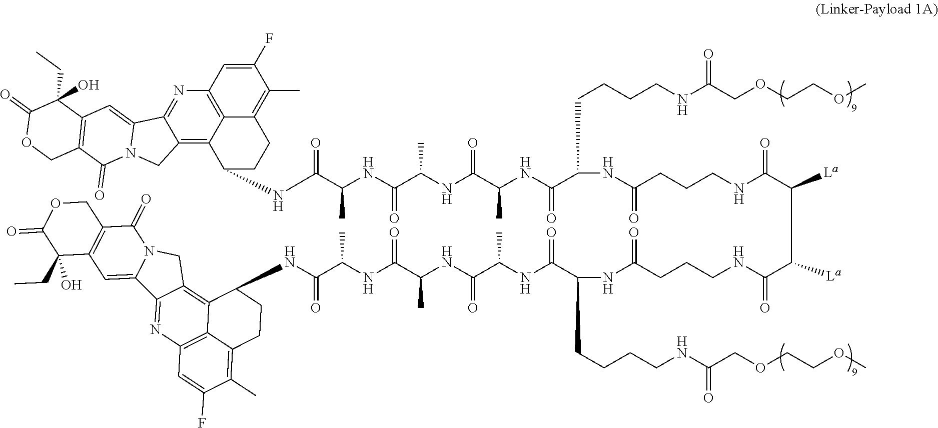

or a stereoisomer thereof, or a pharmaceutically acceptable salt thereof. In some embodiments, each L^{a} is independently

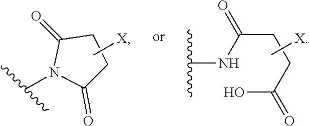

In some embodiments, the polypeptide comprises an amino acid sequence with at least 75%, at least 80%, at least 85%, at least 90%, at least 95%, or at least 99% homology to SEQ ID NO: 1. In some embodiments, the polypeptide comprises an amino acid sequence with at least 75%, at least 80%, at least 85%, at least 90%, at least 95%, or at least 99% homology to SEQ ID NO: 2. In some embodiments, the polypeptide comprises first amino acid sequence with at least 75%, at least 80%, at least 85%, at least 90%, at least 95%, or at least 99% homology to SEQ ID NO: 1; and a second amino acid sequence with at least 75%, at least 80%, at least 85%, at least 90%, at least 95%, or at least 99% homology to SEQ ID NO: 2. In some embodiments, the polypeptide comprises an amino acid sequence with at least 75%, at least 80%, at least 85%, at least 90%, at least 95%, or at least 99% homology to SEQ ID NO: 3. In some embodiments, the polypeptide comprises an amino acid sequence with at least 75%, at least 80%, at least 85%, at least 90%, at least 95%, or at least 99% homology to SEQ ID NO: 4. In some embodiments, the polypeptide comprises a first amino acid sequence with at least 75%, at least 80%, at least 85%, at least 90%, at least 95%, or at least 99% homology to SEQ ID NO: 3; and a second amino acid sequence with at least 75%, at least 80%, at least 85%, at least 90%, at least 95%, or at least 99% homology to SEQ ID NO: 4.

In some embodiments, the polypeptide comprises a heavy chain complementarity determining region 1 (HCDR1) comprising an amino acid sequence with at least 75%, at least 80%, at least 85%, at least 90%, at least 95%, or at least 99% homology to SEQ ID NO: 5. In some embodiments, the polypeptide comprises a heavy chain complementarity determining region 2 (HCDR2) comprising an amino acid sequence with at least 75%, at least 80%, at least 85%, at least 90%, at least 95%, or at least 99% homology to SEQ ID NO: 6. In some embodiments, the polypeptide comprises a heavy chain complementarity determining region 3 (HCDR3) comprising an amino acid sequence that is at least 90%, at least 95%, or at least 99% homology to SEQ ID NO: 7. In some embodiments, the polypeptide comprises a light chain complementarity determining region 1 (LCDR1) comprising an amino acid sequence with at least 75%, at least 80%, at least 85%, at least 90%, at least 95%, or at least 99% homology to SEQ ID NO: 8. In some embodiments, the polypeptide comprises a light chain complementarity determining region 2 (LCDR2) comprising an amino acid sequence with at least 75%, at least 80%, at least 85%, at least 90%, at least 95%, or at least 99% homology to SEQ ID NO: 9. In some embodiments, the polypeptide comprises a light chain complementarity determining region 3 (LCDR3) comprising an amino acid with at least 75%, at least 80%, at least 85%, at least 90%, at least 95%, or at least 99% homology to SEQ ID NO: 10.

In some embodiments, the polypeptide comprises a heavy chain complementarity determining region 1 (HCDR1) comprising an amino acid sequence with at least 75%, at least 80%, at least 85%, at least 90%, at least 95%, or at least 99% homology to SEQ ID NO: 21. In some embodiments, the polypeptide comprises a heavy chain complementarity determining region 2 (HCDR2) comprising an amino acid sequence with at least 75%, at least 80%, at least 85%, at least 90%, at least 95%, or at least 99% homology to SEQ ID NO: 22. In some embodiments, the polypeptide comprises a heavy chain complementarity determining region 3 (HCDR3) comprising an amino acid sequence that is at least 90%, at least 95%, or at least 99% homology to SEQ ID NO: 23. In some embodiments, the polypeptide comprises a light chain complementarity determining region 1 (LCDR1) comprising an amino acid sequence with at least 75%, at least 80%, at least 85%, at least 90%, at least 95%, or at least 99% homology to SEQ ID NO: 24. In some embodiments, the polypeptide comprises a light chain complementarity determining region 2 (LCDR2) comprising an amino acid sequence with at least 75%, at least 80%, at least 85%, at least 90%, at least 95%, or at least 99% homology to SEQ ID NO: 25. In some embodiments, the polypeptide comprises a light chain complementarity determining region 3 (LCDR3) comprising an amino acid with at least 75%, at least 80%, at least 85%, at least 90%, at least 95%, or at least 99% homology to SEQ ID NO: 26. In some embodiments, the polypeptide comprises a HCDR1 comprising an amino acid sequence that is SEQ ID NO: 21; a HCDR2 comprising an amino acid sequence that is SEQ ID NO: 22; a HCDR3 comprising an amino acid sequence that is SEQ ID NO: 23; a LCDR1 comprising an amino acid sequence that is SEQ ID NO: 24; a LCDR2 comprising an amino acid sequence that is SEQ ID NO: 25; and a LCDR3 comprising an amino acid sequence that is SEQ ID NO: 26.

In some embodiments, the polypeptide comprises a heavy chain complementarity determining region 1 (HCDR1) comprising an amino acid sequence with at least 75%, at least 80%, at least 85%, at least 90%, at least 95%, or at least 99% homology to SEQ ID NO: 31. In some embodiments, the polypeptide comprises a heavy chain complementarity determining region 2 (HCDR2) comprising an amino acid sequence with at least 75%, at least 80%, at least 85%, at least 90%, at least 95%, or at least 99% homology to SEQ ID NO: 32. In some embodiments, the polypeptide comprises a heavy chain complementarity determining region 3 (HCDR3) comprising an amino acid sequence that is at least 90%, at least 95%, or at least 99% homology to SEQ ID NO: 33. In some embodiments, the polypeptide comprises a light chain complementarity determining region 1 (LCDR1) comprising an amino acid sequence with at least 75%, at least 80%, at least 85%, at least 90%, at least 95%, or at least 99% homology to SEQ ID NO: 34. In some embodiments, the polypeptide comprises a light chain complementarity determining region 2 (LCDR2) comprising an amino acid sequence with at least 75%, at least 80%, at least 85%, at least 90%, at least 95%, or at least 99% homology to SEQ ID NO: 35. In some embodiments, the polypeptide comprises a light chain complementarity determining region 3 (LCDR3) comprising an amino acid with at least 75%, at least 80%, at least 85%, at least 90%, at least 95%, or at least 99% homology to SEQ ID NO: 36. In some embodiments, the polypeptide comprises a HCDR1 comprising an amino acid sequence that is SEQ ID NO: 31; a HCDR2 comprising an amino acid sequence that is SEQ ID NO: 32; a HCDR3 comprising an amino acid sequence that is SEQ ID NO: 33; a LCDR1 comprising an amino acid sequence that is SEQ ID NO: 34; a LCDR2 comprising an amino acid sequence that is SEQ ID NO: 35; and a LCDR3 comprising an amino acid sequence that is SEQ ID NO: 36.

In some embodiments, the polypeptide comprises a heavy chain complementarity determining region 1 (HCDR1) comprising an amino acid sequence with at least 75%, at least 80%, at least 85%, at least 90%, at least 95%, or at least 99% homology to SEQ ID NO: 41. In some embodiments, the polypeptide comprises a heavy chain complementarity determining region 2 (HCDR2) comprising an amino acid sequence with at least 75%, at least 80%, at least 85%, at least 90%, at least 95%, or at least 99% homology to SEQ ID NO: 42. In some embodiments, the polypeptide comprises a heavy chain complementarity determining region 3 (HCDR3) comprising an amino acid sequence that is at least 90%, at least 95%, or at least 99% homology to SEQ ID NO: 43. In some embodiments, the polypeptide comprises a light chain complementarity determining region 1 (LCDR1) comprising an amino acid sequence with at least 75%, at least 80%, at least 85%, at least 90%, at least 95%, or at least 99% homology to SEQ ID NO: 44. In some embodiments, the polypeptide comprises a light chain complementarity determining region 2 (LCDR2) comprising an amino acid sequence with at least 75%, at least 80%, at least 85%, at least 90%, at least 95%, or at least 99% homology to SEQ ID NO: 45. In some embodiments, the polypeptide comprises a light chain complementarity determining region 3 (LCDR3) comprising an amino acid with at least 75%, at least 80%, at least 85%, at least 90%, at least 95%, or at least 99% homology to SEQ ID NO: 46. In some embodiments, the polypeptide comprises a HCDR1 comprising an amino acid sequence that is SEQ ID NO: 41; a HCDR2 comprising an amino acid sequence that is SEQ ID NO: 42; a HCDR3 comprising an amino acid sequence that is SEQ ID NO: 43; a LCDR1 comprising an amino acid sequence that is SEQ ID NO: 44; a LCDR2 comprising an amino acid sequence that is SEQ ID NO: 45; and a LCDR3 comprising an amino acid sequence that is SEQ ID NO: 46.

In some embodiments, the polypeptide comprises a heavy chain complementarity determining region 1 (HCDR1) comprising an amino acid sequence with at least 75%, at least 80%, at least 85%, at least 90%, at least 95%, or at least 99% homology to SEQ ID NO: 51. In some embodiments, the polypeptide comprises a heavy chain complementarity determining region 2 (HCDR2) comprising an amino acid sequence with at least 75%, at least 80%, at least 85%, at least 90%, at least 95%, or at least 99% homology to SEQ ID NO: 52. In some embodiments, the polypeptide comprises a heavy chain complementarity determining region 3 (HCDR3) comprising an amino acid sequence that is at least 90%, at least 95%, or at least 99% homology to SEQ ID NO: 53. In some embodiments, the polypeptide comprises a light chain complementarity determining region 1 (LCDR1) comprising an amino acid sequence with at least 75%, at least 80%, at least 85%, at least 90%, at least 95%, or at least 99% homology to SEQ ID NO: 54. In some embodiments, the polypeptide comprises a light chain complementarity determining region 2 (LCDR2) comprising an amino acid sequence with at least 75%, at least 80%, at least 85%, at least 90%, at least 95%, or at least 99% homology to SEQ ID NO: 55. In some embodiments, the polypeptide comprises a light chain complementarity determining region 3 (LCDR3) comprising an amino acid with at least 75%, at least 80%, at least 85%, at least 90%, at least 95%, or at least 99% homology to SEQ ID NO: 56. In some embodiments, the polypeptide comprises a HCDR1 comprising an amino acid sequence that is SEQ ID NO: 51; a HCDR2 comprising an amino acid sequence that is SEQ ID NO: 52; a HCDR3 comprising an amino acid sequence that is SEQ ID NO: 53; a LCDR1 comprising an amino acid sequence that is SEQ ID NO: 54; a LCDR2 comprising an amino acid sequence that is SEQ ID NO: 55; and a LCDR3 comprising an amino acid sequence that is SEQ ID NO: 56.

In some embodiments, the polypeptide comprises a heavy chain complementarity determining region 1 (HCDR1) comprising an amino acid sequence with at least 75%, at least 80%, at least 85%, at least 90%, at least 95%, or at least 99% homology to SEQ ID NO: 61. In some embodiments, the polypeptide comprises a heavy chain complementarity determining region 2 (HCDR2) comprising an amino acid sequence with at least 75%, at least 80%, at least 85%, at least 90%, at least 95%, or at least 99% homology to SEQ ID NO: 62. In some embodiments, the polypeptide comprises a heavy chain complementarity determining region 3 (HCDR3) comprising an amino acid sequence that is at least 90%, at least 95%, or at least 99% homology to SEQ ID NO: 63. In some embodiments, the polypeptide comprises a light chain complementarity determining region 1 (LCDR1) comprising an amino acid sequence with at least 75%, at least 80%, at least 85%, at least 90%, at least 95%, or at least 99% homology to SEQ ID NO: 64. In some embodiments, the polypeptide comprises a light chain complementarity determining region 2 (LCDR2) comprising an amino acid sequence with at least 75%, at least 80%, at least 85%, at least 90%, at least 95%, or at least 99% homology to SEQ ID NO: 65. In some embodiments, the polypeptide comprises a light chain complementarity determining region 3 (LCDR3) comprising an amino acid with at least 75%, at least 80%, at least 85%, at least 90%, at least 95%, or at least 99% homology to SEQ ID NO: 66. In some embodiments, the polypeptide comprises a HCDR1 comprising an amino acid sequence that is SEQ ID NO: 61; a HCDR2 comprising an amino acid sequence that is SEQ ID NO: 62; a HCDR3 comprising an amino acid sequence that is SEQ ID NO: 63; a LCDR1 comprising an amino acid sequence that is SEQ ID NO: 64; a LCDR2 comprising an amino acid sequence that is SEQ ID NO: 65; and a LCDR3 comprising an amino acid sequence that is SEQ ID NO: 66. In some embodiments, polypeptide binds to an epitope comprising an amino acid sequence with at least 75%, at least 80%, at least 85%, at least 90%, at least 95%, or at least 99% homology to SEQ ID NO: 11. In some embodiments, the polypeptide binds to PTK7.

Also described herein, in some aspects are methods or uses of utilizing the conjugate described herein. In some embodiments, the method or use comprises delivering a drug to a cell of the subject. In some embodiments, the cell is associated with a disease or condition described herein (e.g., a tumor cell or a cancer cell). In some embodiments, the method or use comprises treating a disease or condition described herein. In some embodiments, the method or use comprises administering the conjugate to a subject in need thereof. In some embodiments, the conjugate targets and delivers the drug to a cell or a site expressing PTK7. In some embodiments, the disease or condition comprises cancer. In some embodiments, the cancer comprises non-small cell lung cancer (NSCLC); ovarian cancer; breast cancer; gastrointestinal cancer; prostate cancer; head and neck cancer; endometrial cancer; cervical cancer; or a combination thereof.

### Conjugates

Provided herein are conjugates of Formula (I): X-[L-D]~
wherein:
n is a number from 1 to 20;
X is a polypeptide or an amino acid residue of a polypeptide;
L is a linker that covalently links X to D; and
D is a drug.

In some embodiments, D comprises a topoisomerase type 1 inhibitor. In some embodiments, the topoisomerase type 1 inhibitor comprises exatecan, topotecan, doxorubicin, irinotecan, urtotecan, diflomotecan, gimatecan, belotecan, edotecarin, topovale, rosettacin, cositecan, etirinotecan pegol, silatecan, 9-aminocamptothecin, elmotecan, or HOCPT. In some embodiments of Formula (I), n is 2, 4, or 6. In some embodiments of Formula (I), n is 6.

In some embodiments, D comprises the structure of Formula (Ia):

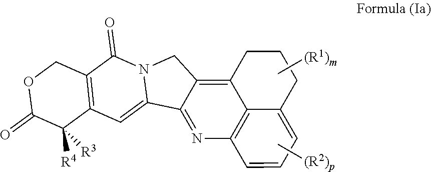

or a stereoisomer thereof, or a pharmaceutically acceptable salt thereof, wherein:
each R¹, R², R³, and R⁴ is independently H, D, F, Cl, Br, I, OH, C₁-C₆ alkyl, C₁-C₆ heteroalkyl, C₁₋₄ alkoxy, C₁₋₄ alkylamino, 5- to 6-membered heteroaryl, 3- to 10-membered heterocycloalkyl, each of which is optionally substituted with 1, 2, or 3 R^{a};
each R^{a} is independently H, D, F, Cl, Br, I, OH, C₁₋₃ alkyl, C₁₋₃ alkoxy, C₁₋₃ alkylamino, CHO, C(O)CH₃, wherein each C₁₋₃ alkyl, C₁₋₃ alkoxy, and C₁₋₃ alkylamino, is optionally substituted with 1, 2 or 3 R;
each R is independently D, F, Cl, Br, I, OH, CH₃, CF₃, NH₂, OCH₃, CHO, C(O)CH₃, or OCF₃; and
m, and p, and are each independently 0, 1, 2, or 3;
wherein each “3- to 6-membered heterocycloalkyl”, “3- to 10-membered heterocycloalkyl”, and “5- or 6-membered heteroaryl” each independently contains 1 or 2 heteroatoms or heteroatom groups independently selected from —NH—, —O—, —S—, and N.

In some embodiments of Formula (Ia), m is 1. In some embodiments of Formula (Ia), p is 2. In some embodiments of Formula (Ia), m is 1 and p is 2. In some embodiments of Formula (Ia), R² is F. In some embodiments of Formula (Ia), R² is CH₃. In some embodiments of Formula (Ia), R² is CH₃. In some embodiments of Formula (Ia), R¹ is NH₂. In some embodiments of Formula (Ia), R⁴ is CH₂CH₃. In some embodiments of Formula (Ia), R³ is OH. In some embodiments of Formula (Ia), X is attached to L via R¹. In some embodiments of Formula (Ia), D comprises the structure:

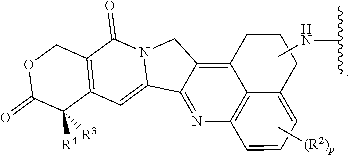

In some embodiments of Formula (Ia), D comprises the structure:

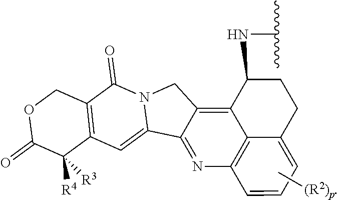

In some embodiments of Formula (Ia), D comprises the structure:

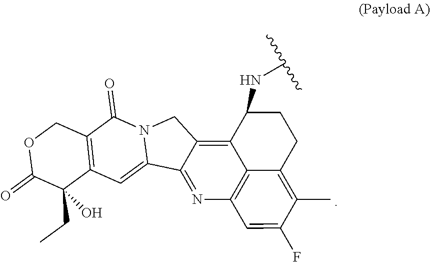 [SMILES: *N[C@H]1CCc2c(C)c(F)cc3nc4c(c1c23)Cn1c-4cc2c(c1=O)COC(=O)[C@]2(O)CC]

In some embodiments, each L independently comprises the structure of Formula (Ib):

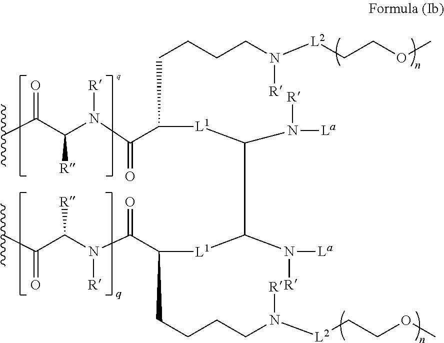

or a stereoisomer thereof, or a pharmaceutically acceptable salt thereof, wherein:
each R″ is independently hydrogen or C₁₋₆ alkyl, wherein the C₁₋₆ alkyl is optionally substituted with phenyl, —C(═O)NR′₂, —C(═O)OR′, —OR′, —NR′₂, —SR′, —NR′C(═O)NR′₂, or —NR′C(═NH)NR′₂; and
each R′ is independently —H or —CH₃;
each n is 1-15;
each q is 1-3;
each L¹ is —NR′C(O)(CH₂)₂₋₄—NR′C(O)—, —C(O)NR′(CH₂)₂₋₄—C(O)NR′—, —(CH₂)₁₋₆—, —OC(O)(CH₂)₂₋₄—NR′C(O)—, or —NR′C(O)(CH₂)₂₋₄—OC(O)—;
each L² is —C(O)(CH₂)₁₋₃O—, —C(O)(CH₂)₁₋₃NR′—, —C(O)NR′(CH₂)₁₋₃O—, or —(CH₂)₁₋₃—; and
each L is independently

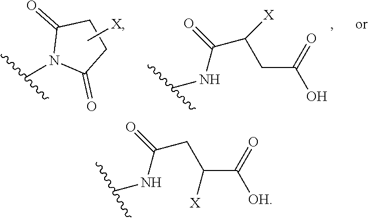

In some embodiments of Formula (Ib), q is 3. In some embodiments of Formula (Ib), n is 8-10. In some embodiments of Formula (Ib), n is 9. In some embodiments of Formula (Ib), R′ is H. In some embodiments of Formula (Ib), L¹ is —NR′C(O)(CH₂)₂₋₄—NR′C(O)—. In some embodiments of Formula (Ib), L¹ is —NR′C(O)(CH₂)₃—NR′C(O)—. In some embodiments of Formula (Ib), L² is —C(O)(CH₂)₁₋₃O—. In some embodiments of Formula (Ib), L² is —C(O)(CH₂)O—.

In some embodiments, each L independently comprises the structure of Formula (Ic):

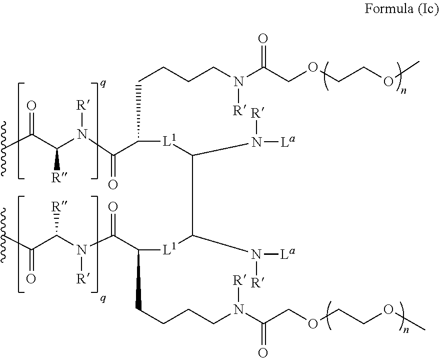

or a stereoisomer thereof, or a pharmaceutically acceptable salt thereof, wherein:
each R″ is independently hydrogen or C₁₋₆ alkyl, wherein the C₁₋₆ alkyl is optionally substituted with phenyl, —C(═O)NR′₂, —C(═O)OR′, —OR′, —NR′₂, —SR′, —NR′C(═O)NR′₂, or —NR′C(═NH)NR′₂; and
each R′ is independently —H or —CH₃;
each n is 1-15;
each q is 1-3;
each L¹ is —NR′C(O)(CH₂)₂₋₄—NR′C(O)—, —C(O)NR′(CH₂)₂₋₄—C(O)NR′—, —(CH₂)₁₋₆—, —OC(O)(CH₂)₂₋₄—NR′C(O)—, or —NR′C(O)(CH₂)₂₋₄—OC(O)—;
and
each L is independently

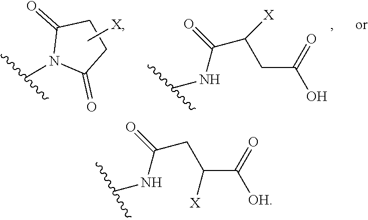

In some embodiments of Formula (Ic), q is 3. In some embodiments of Formula (Ic), n is 8-10. In some embodiments of Formula (Ic), n is 9. In some embodiments of Formula (Ic), R′ is H. In some embodiments of Formula (Ic), L¹ is —NR′C(O)(CH₂)₂₋₄—NR′C(O)—. In some embodiments of Formula (Ic), L¹ is —NR′C(O)(CH₂)₃—NR′C(O)—.

In some embodiments, each L independently comprises the structure of Formula (Id):

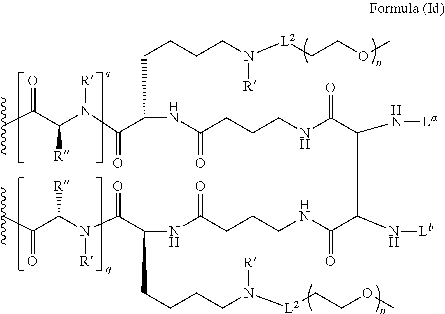

or a stereoisomer thereof, or a pharmaceutically acceptable salt thereof, wherein:
each R″ is independently hydrogen or C₁₋₆ alkyl, wherein the C₁₋₆ alkyl is optionally substituted with phenyl, —C(═O)NR′₂, —C(═O)OR′, —OR′, —NR′₂, —SR′, —NR′C(═O)NR′₂, or —NR′C(═NH)NR′₂, and
each R′ is independently —H or —CH₃;
each n is 1-15;
each q is 1-3;
each L² is —C(O)(CH₂)₁₋₃O—, —C(O)(CH₂)₁₋₃NR′—, —C(O)NR′(CH₂)₁₋₃O—, or —(CH₂)₁₋₃—;
and
each L^{a} is independently

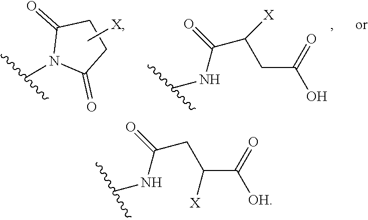

In some embodiments of Formula (Ic), q is 3. In some embodiments of Formula (Ic), n is 8-10. In some embodiments of Formula (Ic), n is 9. In some embodiments of Formula (Ic), R′ is H. In some embodiments of Formula (Ic), L² is —C(O)(CH₂)₁₋₃O—. In some embodiments of Formula (Ic), L² is —C(O)(CH₂)O—.

In some embodiments, each L independently comprises the structure of Formula (Ie):

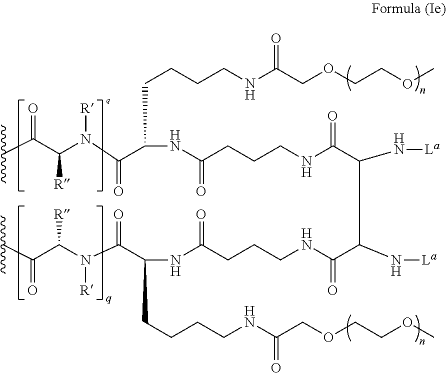

or a stereoisomer thereof, or a pharmaceutically acceptable salt thereof, wherein:
each R″ is independently hydrogen or C₁₋₆ alkyl, wherein the C₁₋₆ alkyl is optionally substituted with phenyl, —C(═O)NR′₂, —C(═O)OR′, —OR′, —NR′₂, —SR′, —NR′C(═O)NR′₂, or —NR′C(═NH)NR′₂; and
each R′ is independently —H or —CH₃;
each n is 1-15;
each q is 1-3; and
each L^{a} is independently

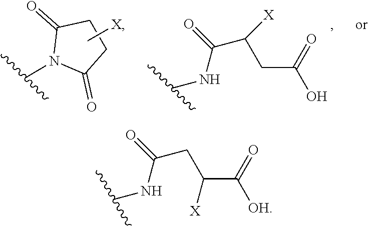

In some embodiments of Formulas (Ib-Ie),

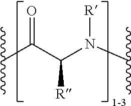

comprises

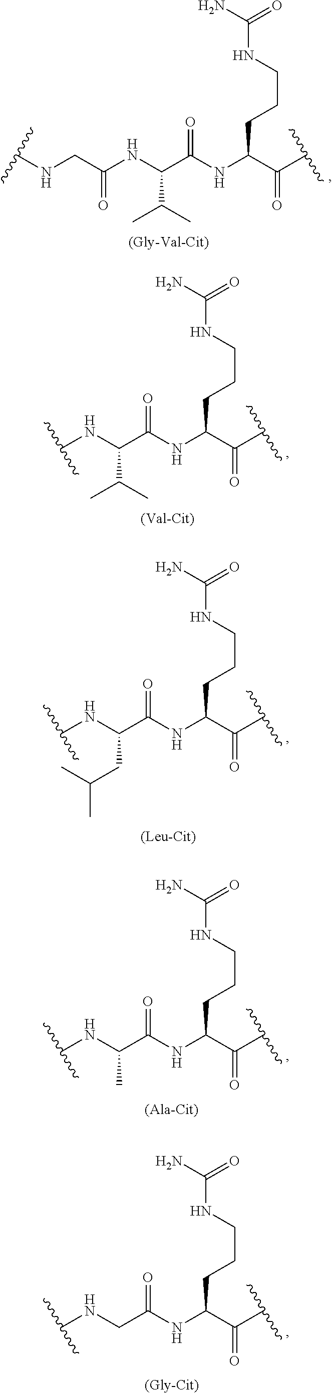 [SMILES: *NCC(=O)N[C@H](C(=O)N[C@@H](CCCNC(N)=O)C(*)=O)C(C)C ; *N[C@H](C(=O)N[C@@H](CCCNC(N)=O)C(*)=O)C(C)C ; *N[C@@H](CC(C)C)C(=O)N[C@@H](CCCNC(N)=O)C(*)=O ; *N[C@@H](C)C(=O)N[C@@H](CCCNC(N)=O)C(*)=O ; *NCC(=O)N[C@@H](CCCNC(N)=O)C(*)=O]

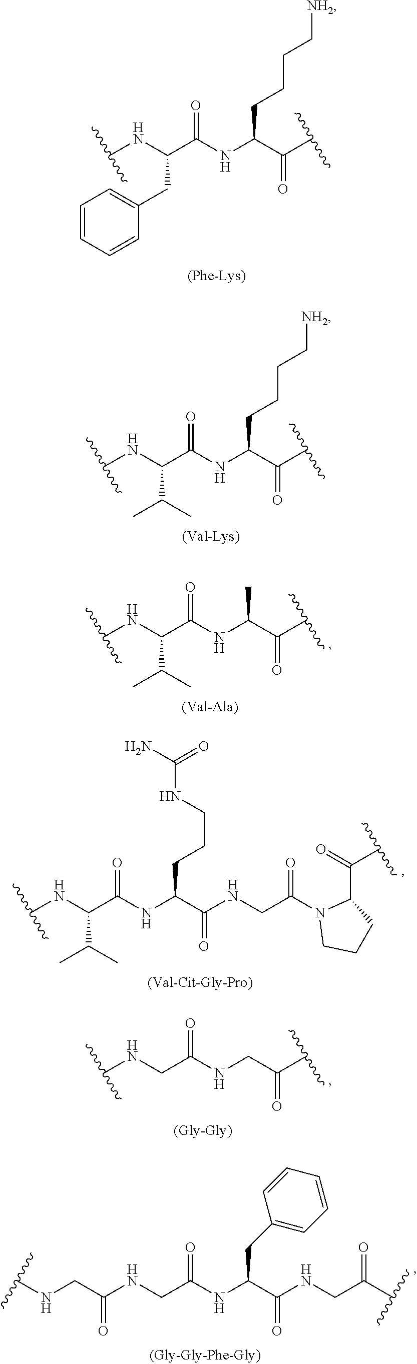 [SMILES: *N[C@@H](Cc1ccccc1)C(=O)N[C@@H](CCCCN)C(*)=O ; *N[C@H](C(=O)N[C@@H](CCCCN)C(*)=O)C(C)C ; *N[C@H](C(=O)N[C@@H](C)C(*)=O)C(C)C ; *N[C@H](C(=O)N[C@@H](CCCNC(N)=O)C(=O)NCC(=O)N1CCC[C@H]1C(*)=O)C(C)C ; *NCC(=O)NCC(*)=O ; *NCC(=O)NCC(=O)N[C@@H](Cc1ccccc1)C(=O)NCC(*)=O]

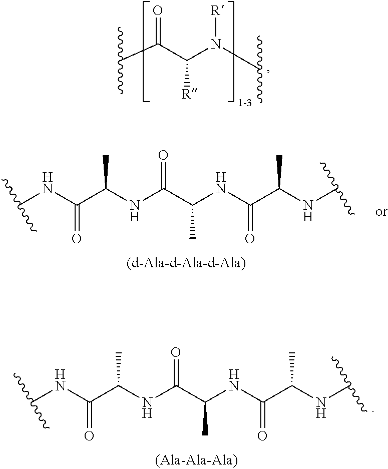

In some embodiments, each L independently comprises the structures:

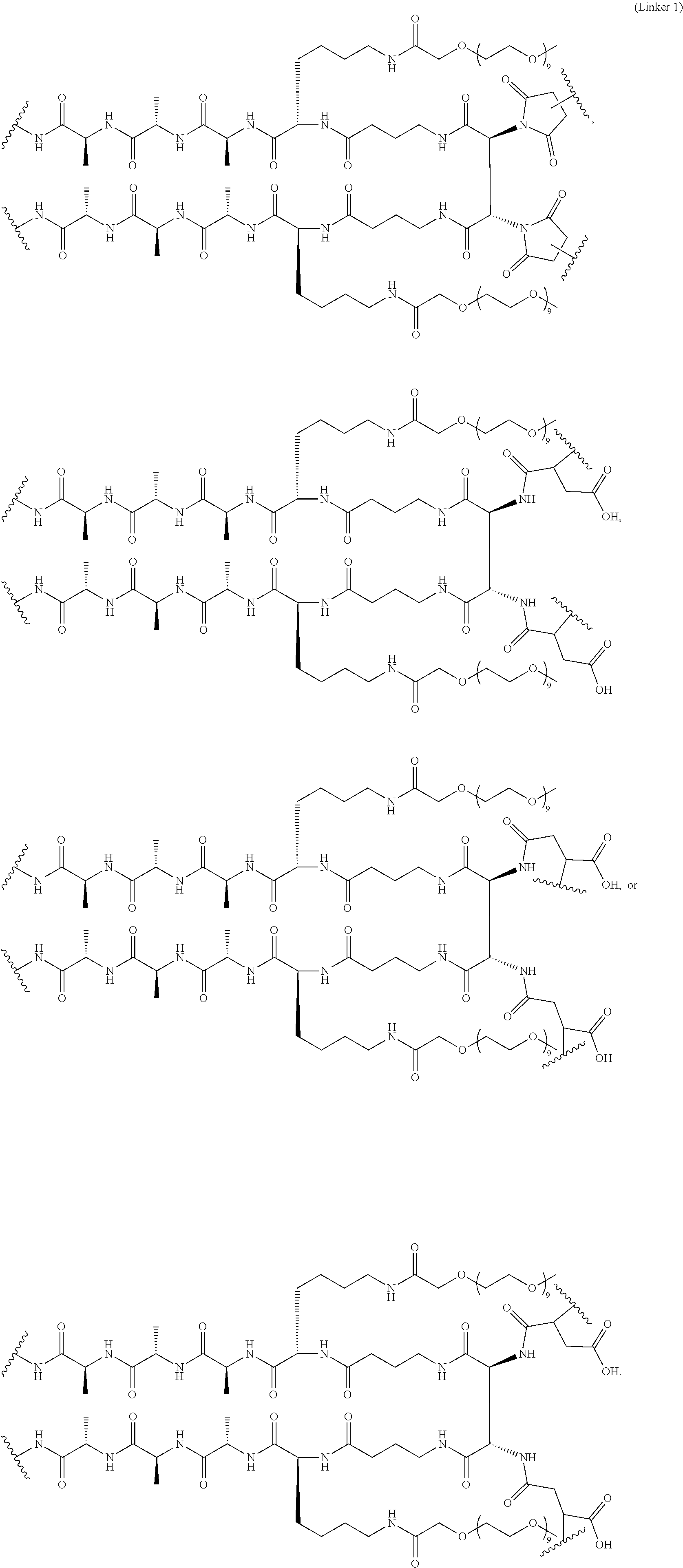

### Antibodies

The conjugates of the present disclosure can comprise an antibody or antigen binding fragment thereof. The linker payload (e.g., L-D) described herein may be conjugated to a suitable antibody or antigen binding fragment thereof or targeting polypeptide.

In some embodiments, the term “antibody” is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (for example, sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, for example, bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (HCDR1, HCDR2, or HCDR3) and three CDRs in each light chain variable region (LCDR1, LCDR2, or LCDR3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745. (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme). The CDRs of the antibodies described herein may be defined by the Kabat, IMGT, Chothia, AbM, Aho, contact numbering scheme, or any combination thereof.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See for example, Kindt et al. Kuby Immunology, 6th ed., W.H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See for example, Portolano et al., J. Immunol. 150:880-887 (1993); Clarkson et al., Nature 352:624-628 (1991)).

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while p and F contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (x) or lambda (k) based on the amino acid sequence of the constant domains. Light chain amino acid sequences are well known in the art. A light chain can be a human light chain.

Among the provided antibodies are antibody fragments. An “antibody fragment,” “antigen-binding fragment,” “antigen-binding domain,” “antigen-binding region,” “antigen binding fragment,” “antigen binding domain,” “antigen binding region,” and similar terms refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (for example, scFv or sFv); and multispecific antibodies formed from antibody fragments. In particular embodiments, the antibodies are single-chain antibody fragments comprising a variable heavy chain region and/or a variable light chain region, such as scFvs. Generally, an antibody fragment or antigen-binding fragment will comprise one or more CDRs from a parental antibody that are sufficient to confer binding specificity.

Generally, a humanized antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (for example, the antibody from which the CDR residues are derived), for example, to restore or improve antibody specificity or affinity. In some embodiments, a humanized antibody refers to forms of non-human (for example, murine) or not fully humanized antibodies having specific immunoglobulin chains, chimeric immunoglobulins, or fragments thereof that contain minimal non-human (for example, murine) sequences.

In some embodiments, an antibody, antigen binding fragment thereof, or polypeptide provided herein has a dissociation constant (K_{D}) of about 1 μm, 100 nM, 50 nM, 40 nM, 30 nM, 20 nM, 10 nM, 5 nM, 2 nM, 1 nM, 0.5 nM, 0.1 nM, 0.05 nM, 0.01 nM or less (e.g., 10⁻⁸ M or less, e.g., from 10⁻⁸ M to 10⁻¹³ M, e.g., from 10⁻⁹ M to 10⁻¹³ M) for the antibody target. In some embodiments, an antibody provided herein has a dissociation constant (K_{D}) of about 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or greater (e.g., 10⁻⁸ M or less, e.g., from 10⁻⁸ M to 10⁻¹³ M, e.g., from 10⁻⁹ M to 10⁻¹³ M) for the antibody target. K_{D} can be measured, for example, using surface plasmon resonance assays (e.g., using a BIACORE®-2000, a BIACORE®-3000 or Octet).

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived or selected from human antibody libraries, including phage display and cell-free libraries, containing antibody-encoding sequences derived from a human repertoire. In certain embodiments, a human antibody can have sequence liabilities removed or its affinity increased by successive rounds of selection by a method such as phage display.

The antibodies, antigen binding fragments thereof, or polypeptides described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large scale cell culture are known in the art. See e.g., Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NS0 murine myeloma cell, or a PER.C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

In some embodiments, the polypeptide (e.g., antibody or antigen binding fragment thereof described herein) comprises the polypeptide comprises an amino acid sequence with at least 75%, at least 80%, at least 85%, at least 90%, at least 95%, or at least 99% homology to SEQ ID NO: 1. In some embodiments, the polypeptide comprises an amino acid sequence with at least 75%, at least 80%, at least 85%, at least 90%, at least 95%, or at least 99% homology to SEQ ID NO: 2. In some embodiments, the polypeptide comprises first amino acid sequence with at least 75%, at least 80%, at least 85%, at least 90%, at least 95%, or at least 99% homology to SEQ ID NO: 1; and a second amino acid sequence with at least 75%, at least 80%, at least 85%, at least 90%, at least 95%, or at least 99% homology to SEQ ID NO: 2. In some embodiments, the polypeptide comprises a first amino acid sequence that is SEQ ID NO: 1; and a second amino acid sequence that is SEQ ID NO: 2. In some embodiments, the polypeptide comprises an amino acid sequence that is at least 50 contiguous amino, at least contiguous 100 amino acids, at least 150 contiguous amino, or at least contiguous 200 amino acids homology to SEQ ID NO: 1. In some embodiments, the polypeptide comprises an amino acid sequence that is at least 50 contiguous amino, at least contiguous 100 amino acids, at least 150 contiguous amino, or at least contiguous 200 amino acids homology to SEQ ID NO: 2. In some embodiments, the polypeptide comprises a first amino acid sequence that is at least 50 contiguous amino, at least contiguous 100 amino acids, at least 150 contiguous amino, or at least contiguous 200 amino acids homology to SEQ ID NO: 1; and a second amino acid sequence that is at least 50 contiguous amino, at least contiguous 100 amino acids, at least 150 contiguous amino, or at least contiguous 200 amino acids homology to SEQ ID NO: 2. In some embodiments, the polypeptide comprises an amino acid sequence with at least 75%, at least 80%, at least 85%, at least 90%, at least 95%, or at least 99% homology to SEQ ID NO: 3. In some embodiments, the polypeptide comprises an amino acid sequence with at least 75%, at least 80%, at least 85%, at least 90%, at least 95%, or at least 99% homology to SEQ ID NO: 4. In some embodiments, the polypeptide comprises a first amino acid sequence with at least 75%, at least 80%, at least 85%, at least 90%, at least 95%, or at least 99% homology to SEQ ID NO: 3; and a second amino acid sequence with at least 75%, at least 80%, at least 85%, at least 90%, at least 95%, or at least 99% homology to SEQ ID NO: 4. In some embodiments, the polypeptide comprises a first amino acid sequence that is SEQ ID NO: 3; and a second amino acid sequence that is SEQ ID NO: 4. In some embodiments, the polypeptide comprises an amino acid sequence that is at least 50 contiguous amino acids or at least contiguous 100 amino acids homology to SEQ ID NO: 3. In some embodiments, the polypeptide comprises an amino acid sequence that is at least 50 contiguous amino acids or at least contiguous 100 amino acids homology to SEQ ID NO: 4.

In some embodiments, the polypeptide comprises a heavy chain complementarity determining region 1 (HCDR1) comprising an amino acid sequence with at least 75%, at least 80%, at least 85%, at least 90%, at least 95%, or at least 99% homology to SEQ ID NO: 5. In some embodiments, the polypeptide comprises a heavy chain complementarity determining region 2 (HCDR2) comprising an amino acid sequence with at least 75%, at least 80%, at least 85%, at least 90%, at least 95%, or at least 99% homology to SEQ ID NO: 6. In some embodiments, the polypeptide comprises a heavy chain complementarity determining region 3 (HCDR3) comprising an amino acid sequence that is at least 75%, at least 80%, at least 85%, at least 90%, at least 95%, or at least 99% homology to SEQ ID NO: 7. In some embodiments, the polypeptide comprises a light chain complementarity determining region 1 (LCDR1) comprising an amino acid sequence with at least 75%, at least 80%, at least 85%, at least 90%, at least 95%, or at least 99% homology to SEQ ID NO: 8. In some embodiments, the polypeptide comprises a light chain complementarity determining region 2 (LCDR2) comprising an amino acid sequence with at least 75%, at least 80%, at least 85%, at least 90%, at least 95%, or at least 99% homology to SEQ ID NO: 9. In some embodiments, the polypeptide comprises a light chain complementarity determining region 3 (LCDR3) comprising an amino acid with at least 75%, at least 80%, at least 85%, at least 90%, at least 95%, or at least 99% homology to SEQ ID NO: 10. In some embodiments, the polypeptide comprises a HCDR1 comprising an amino acid sequence that is SEQ ID NO: 5; a HCDR2 comprising an amino acid sequence that is SEQ ID NO: 6; a HCDR3 comprising an amino acid sequence that is SEQ ID NO: 7; a LCDR1 comprising an amino acid sequence that is SEQ ID NO: 8; a LCDR2 comprising an amino acid sequence that is SEQ ID NO: 9; and a LCDR3 comprising an amino acid sequence that is SEQ ID NO: 10. In some embodiments, the polypeptide binds to an epitope comprising an amino acid sequence with at least 75%, at least 80%, at least 85%, at least 90%, at least 95%, or at least 99% homology to SEQ ID NO: 11. In some embodiments, the polypeptide binds to PTK7.

In some embodiments, the polypeptide comprises a HCDR1 comprising an amino acid sequence that is SEQ ID NO: 21; a HCDR2 comprising an amino acid sequence that is SEQ ID NO: 22; a HCDR3 comprising an amino acid sequence that is SEQ ID NO: 23; a LCDR1 comprising an amino acid sequence that is SEQ ID NO: 24; a LCDR2 comprising an amino acid sequence that is SEQ ID NO: 25; and a LCDR3 comprising an amino acid sequence that is SEQ ID NO: 26. In some embodiments, the polypeptide comprises a HCDR1 comprising an amino acid sequence that is SEQ ID NO: 31; a HCDR2 comprising an amino acid sequence that is SEQ ID NO: 32; a HCDR3 comprising an amino acid sequence that is SEQ ID NO: 33; a LCDR1 comprising an amino acid sequence that is SEQ ID NO: 34; a LCDR2 comprising an amino acid sequence that is SEQ ID NO: 35; and a LCDR3 comprising an amino acid sequence that is SEQ ID NO: 36. In some embodiments, the polypeptide comprises a HCDR1 comprising an amino acid sequence that is SEQ ID NO: 41; a HCDR2 comprising an amino acid sequence that is SEQ ID NO: 42; a HCDR3 comprising an amino acid sequence that is SEQ ID NO: 43; a LCDR1 comprising an amino acid sequence that is SEQ ID NO: 44; a LCDR2 comprising an amino acid sequence that is SEQ ID NO: 45; and a LCDR3 comprising an amino acid sequence that is SEQ ID NO: 46. In some embodiments, the polypeptide comprises a HCDR1 comprising an amino acid sequence that is SEQ ID NO: 51; a HCDR2 comprising an amino acid sequence that is SEQ ID NO: 52; a HCDR3 comprising an amino acid sequence that is SEQ ID NO: 53; a LCDR1 comprising an amino acid sequence that is SEQ ID NO: 54; a LCDR2 comprising an amino acid sequence that is SEQ ID NO: 55; and a LCDR3 comprising an amino acid sequence that is SEQ ID NO: 56. In some embodiments, the polypeptide comprises a HCDR1 comprising an amino acid sequence that is SEQ ID NO: 61; a HCDR2 comprising an amino acid sequence that is SEQ ID NO: 62; a HCDR3 comprising an amino acid sequence that is SEQ ID NO: 63; a LCDR1 comprising an amino acid sequence that is SEQ ID NO: 64; a LCDR2 comprising an amino acid sequence that is SEQ ID NO: 65; and a LCDR3 comprising an amino acid sequence that is SEQ ID NO: 66. In some embodiments, polypeptide binds to an epitope comprising an amino acid sequence with at least 75%, at least 80%, at least 85%, at least 90%, at least 95%, or at least 99% homology to SEQ ID NO: 11. In some embodiments, the polypeptide binds to PTK7.

In some embodiments, the polypeptide comprises a heavy chain complementarity determining region 1 (HCDR1) comprising an amino acid sequence of X₁X₂X₃X₄X₅X₆X₇X₈X₉X₁₀X₁₁X₁₂X₁₃, where:
X₁ comprises G or L;
X₂ comprises E, F, or Y;
X₃ comprises D, E, I, R, S, or T;
X₄ comprises F, G, I, or L;
X₅ comprises S, T, or Y;
X₆ comprises D, G, I, K, N, R, S, or T;
X₇ comprises D, F, S, or Y;
X₈ comprises A, G, L, N, P, T, V, W, or Y;
X₉ comprises A, F, I, M, or V;
X₁₀ comprises E, G, H, Q, S, W, or Y;
X₁₁ comprises N, V, W, or absent;
X₁₂ comprises G, I, or absent; or
X₁₃ comprises E or absent. In some embodiments, the polypeptide comprises a heavy chain complementarity determining region 1 (HCDR1) comprising an amino acid sequence with at least 75%, at least 80%, at least 85%, at least 90%, at least 95%, or at least 99% homology to any one of SEQ ID NOs: 71-86. In some embodiments, the polypeptide comprises a HCDR1 comprising an amino acid sequence that is anyone of SEQ ID NOs: 71-86.

In some embodiments, the polypeptide comprises a heavy chain complementarity determining region 2 (HCDR2) comprising an amino acid sequence of X₁X₂X₃X₄X₅X₆X₇X₈X₉X₁₀X₁₁X₁₂X₁₃X₁₄X₁₅X₁₆X₁₇X₁₈, where
X₁ comprises A, D, E, H, I, S, V, or Y;
X₂ comprises I, L, or Y;
X₃ comprises G, I, L, N, S, W, or Y;
X₄ comprises D, G, N, P, S, T, W, or Y;
X₅ comprises D, G, P, W, or Y;
X₆ comprises D, F, G, N, R, S, T, or Y;
X₇ comprises D, E, G, N, R, or S;
X₈ comprises A, D, G, I, K, N, R, S, T, or Y;
X₉ comprises G, I, K, N, S, T, V, or Y;
X₁₀ comprises D, K, N, P, R, T, or Y;
X₁₁ comprises A, K, N, S, or Y;
X₁₂ comprises A, D, F, L, N, P, T, or V;
X₁₃ comprises D, E, K, P, Q, S, or T;
X₁₄ comprises D, E, K, L, S, or V;
X₁₅ comprises F, K, L, V, or absent;
X₁₆ comprises F, G, K, R, S, or absent;
X₁₇ comprises D, G, K, or absent; or
X₁₈ comprises G, or absent. In some embodiments, the polypeptide comprises a heavy chain complementarity determining region 2 (HCDR2) comprising an amino acid sequence with at least 75%, at least 80%, at least 85%, at least 90%, at least 95%, or at least 99% homology to any one of SEQ ID NOs: 91-106. In some embodiments, the polypeptide comprises a HCDR2 comprising an amino acid sequence that is anyone of SEQ ID NOs: 91-106.

In some embodiments, the polypeptide comprises a heavy chain complementarity determining region 3 (HCDR3) comprising an amino acid sequence of X₁X₂X₃X₄X₅X₆X₇X₈X₉X₁₀X₁₁X₁₂X₁₃X₁₄X₁₅X₁₆X₁₇, where
X₁ comprises D, E, G, I, N, P, R, S, T, or W;
X₂ comprises D, G, K, L, N, T, V, or Y;
X₃ comprises A, G, L, P, Q, S, T, W, or Y;
X₄ comprises D, F, G, H, L, P, S, T, or Y;
X₅ comprises A, D, F, G, I, N, R, V, Y, or absent;
X₆ comprises A, D, G, L, P, S, V, Y, or absent;
X₇ comprises A, D, F, G, M, N, R, W, Y, or absent;
X₈ comprises A, D, F, L, R, S, T, Y, or absent;
X₉ comprises A, D, F, M, S, Y, or absent;
X₁₀ comprises D, G, M, N, T, Y, or absent;
X₁₁ comprises D, F, S, Y, or absent;
X₁₂ comprises A, Y, or absent;
X₁₃ comprises F, Y, or absent;
X₁₄ comprises D, G, or absent;
X₁₅ comprises T, Y, or absent;
X₁₆ comprises D, or absent; or
X₁₇ comprises V, or absent. In some embodiments, the polypeptide comprises a heavy chain complementarity determining region 3 (HCDR3) comprising an amino acid sequence with at least 75%, at least 80%, at least 85%, at least 90%, at least 95%, or at least 99% homology to any one of SEQ ID NOs: 111-129. In some embodiments, the polypeptide comprises a HCDR3 comprising an amino acid sequence that is anyone of SEQ ID NOs: 111-129.

In some embodiments, the polypeptide comprises a light chain complementarity determining region 1 (LCDR1) comprising an amino acid sequence of X₁X₂X₃X₄X₅X₆X₇X₈X₉X₁₀X₁₁X₁₂X₁₃X₁₄X₁₅X₁₆X₁₇X₁₈X₁₉, where
X₁ comprises I, K, or R;
X₂ comprises A, S, or T;
X₃ comprises N or S;
X₄ comprises D, E, Q, or T;
X₅ comprises D, G, H, N, S, or T;
X₆ comprises I, L, or V;
X₇ comprises D, G, L, N, S, or Y;
X₈ comprises D, I, N, P, S, T, or Y;
X₉ comprises D, F, G, N, S, W, or Y;
X₁₀ comprises A, G, L, M, V, or Y;
X₁₁ comprises A, H, I, K, L, M, N, or V;
X₁₂ comprises A, Q, S, T, W, or absent;
X₁₃ comprises F, Q, Y, or absent;
X₁₄ comprises M, N, Q, or absent;
X₁₅ comprises H, N, Q, S, or absent;
X₁₆ comprises K, L, or absent;
X₁₇ comprises A, Q, or absent;
X₁₈ comprises L, or absent; or
X₁₉ comprises A, or absent. In some embodiments, the polypeptide comprises a light chain complementarity determining region 1 (LCDR1) comprising an amino acid sequence with at least 75%, at least 80%, at least 85%, at least 90%, at least 95%, or at least 99% homology to any one of SEQ ID NOs: 131-148. In some embodiments, the polypeptide comprises a LCDR1 comprising an amino acid sequence that is anyone of SEQ ID NOs: 131-148.

In some embodiments, the polypeptide comprises a light chain complementarity determining region 2 (LCDR2) comprising an amino acid sequence of X₁X₂X₃X₄X₅X₆X₇, where
X₁ comprises A, D, E, G, N, R, S, or Y;
X₂ comprises A, G, or T;
X₃ comprises N, S, or T;
X₄ comprises G, N, Q, R, S, T, or Y;
X₅ comprises L, R, or S;
X₆ comprises A, D, E, F, G, I, L, Q, R, or Y; or
X₇ comprises D, E, P, S, or T. In some embodiments, the polypeptide comprises a light chain complementarity determining region 2 (LCDR2) comprising an amino acid sequence with at least 75%, at least 80%, at least 85%, at least 90%, at least 95%, or at least 99% homology to any one of SEQ ID NOs: 151-168. In some embodiments, the polypeptide comprises a LCDR2 comprising an amino acid sequence that is anyone of SEQ ID NOs: 151-168.

In some embodiments, the polypeptide comprises a light chain complementarity determining region 3 (LCDR3) comprising an amino acid sequence of X₁X₂X₃X₄X₅X₆X₇X₈X₉X₁₀, where
X₁ comprises H, L, or Q;
X₂ comprises H, N, or Q;
X₃ comprises C, D, F, H, R, S, or Y;
X₄ comprises D, F, G, H, I, K, N, S, W, or Y;
X₅ comprises E, G, I, N, R, S, T, or absent;
X₆ comprises D, F, I, L, S, T, V, W, Y, or absent;
X₇ comprises P or absent;
X₈ comprises F, I, L, M, P, R, W, Y, or absent;
X₉ comprises F, T, Y, or absent; or
X₁₀ comprises T or absent. In some embodiments, the polypeptide comprises a light chain complementarity determining region 3 (LCDR3) comprising an amino acid sequence with at least 75%, at least 80%, at least 85%, at least 90%, at least 95%, or at least 99% homology to any one of SEQ ID NOs: 171-188. In some embodiments, the polypeptide comprises a LCDR3 comprising an amino acid sequence that is anyone of SEQ ID NOs: 171-188.

### Fc Constant Regions

Generally, the fragment crystallizable (Fc) region or domain of an antibody mediates downstream effector functions via its interaction with Fc-receptors on immune cells (for example, innate immune cells) or with complement protein C1q, the recognition molecule of the complement system. Furthermore, the interaction with Fc-receptors can lead to killing of targeted cells through a variety of immune effector mechanisms, including antibody-dependent cell-mediated cytotoxicity (ADCC) and antibody-dependent cellular phagocytosis (ADCP), and antibody-mediated complement activation may lead to complement-dependent cytotoxicity (CDC). In addition, both Fc-receptor interactions and complement activation can exert a broad range of immunomodulatory functions.

Accordingly, in certain instances, mutations within the Fc region that reduce, inhibit, ablate, and/or abrogate Fc-mediated effector function are advantageous for reducing immune activation resulting from the binding of an antibody to the target. Other mutations may be introduced in the Fc region of an antibody to increase binding to the neonatal Fc receptor. Such binding may serve to increase the serum half-life of the antibody. In some embodiments, one or more amino acid modifications may be introduced into the Fc region of an antibody, thereby generating an Fc region variant. An Fc region may comprise a C-terminal region of an immunoglobulin heavy chain that comprises a hinge region, CH2 domain, CH3 domain, or any combination thereof. As used herein, an Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution, addition, or deletion) at one or more amino acid positions. In some embodiments, the Fc region comprises a human IgG1 Fc region. In some embodiments, the Fc region comprises a human IgG1 Fc region. In some embodiments, the Fc region comprises at least one modification. In some embodiments, the at least one modification comprises a L234A (Kabat numbering) mutation or a L235A (Kabat numbering) mutation. In some embodiments, the at least one modification comprises the L234A (Kabat numbering) mutation and the L235A (Kabat numbering) mutation.

### Pharmaceutical Compositions

In some embodiments, the conjugates disclosed herein and/or encompassed by any of the Formula provided herein, are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, provided herein is a pharmaceutical composition comprising a conjugate of Formula (I) herein, or a conjugate specifically disclosed herein, or a pharmaceutically acceptable salt thereof; or a stereoisomer or mixture of stereoisomers thereof; and a pharmaceutically acceptable excipient. In some embodiments, the pharmaceutical composition comprises a conjugate of Formula (I), and a pharmaceutically acceptable excipient. In some embodiments, the pharmaceutical composition is formulated for administering intratumorally, intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, or a combination thereof. In some embodiments, the pharmaceutical composition is for treating a disease or condition. In some embodiments, the disease or condition comprises solid tumor. In some embodiments, the disease or condition comprises cancer. In some embodiments, the cancer comprises non-small cell lung cancer (NSCLC); ovarian cancer; breast cancer; gastrointestinal cancer; prostate cancer; head and neck cancer; endometrial cancer; cervical cancer; or a combination thereof.

In some embodiments, provided herein is a pharmaceutical composition comprising a conjugate of Formula (I) herein, or a conjugate specifically disclosed herein, or a pharmaceutically acceptable salt thereof, or a stereoisomer or mixture of stereoisomers thereof, and a pharmaceutically acceptable excipient, for use in a method of treating a disease, disorder, or condition. In some embodiments, the disease, disorder, or condition is a cancer and/or tumor.

As used herein, the term “pharmaceutically acceptable” generally refers to approved or approvable by a regulatory agency of the Federal or a state government or listed in the U.S. Pharmacopeia or other generally recognized pharmacopeia, as being safe and/or fit for use in animals, including humans, in the context of medicinal formulations. By “pharmaceutically acceptable,” it is also meant that the carrier, diluent, or excipient must be compatible with the other ingredients of the formulation and not deleterious to the recipient thereof.

As used herein, the term “pharmaceutically acceptable salt,” generally refers to a salt which does not abrogate the biological activity and properties of the compounds of the invention, and does not cause significant irritation to a subject to which it is administered. Examples of such salts include, but are not limited to: (a) acid addition salts formed with inorganic acids, for example, hydrochloric acid, hydrobromic acid, sulfuric acid, phosphoric acid, nitric acid and the like; and salts formed with organic acids, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine. See, e.g., Haynes et al., “Commentary: Occurrence of Pharmaceutically Acceptable Anions and Cations in the Cambridge Structural Database,” J. Pharmaceutical Sciences, vol. 94, no. 10 (2005), and Berge et al., “Pharmaceutical Salts,” J. Pharmaceutical Sciences, vol. 66, no. 1 (1977), which are incorporated by reference herein.

As used herein, the term “pharmaceutically acceptable excipient” or “excipient” generally refers to an inert substance added to a pharmaceutical composition to further facilitate administration of an active ingredient, including carriers, diluents, and/or adjuvants. Formulations for parenteral administration can, for example, contain excipients such as sterile water or saline, polyalkylene glycols such as polyethylene glycol, vegetable oils, or hydrogenated napthalenes. Other exemplary excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20.

In certain embodiments the conjugates of the current disclosure are included in a pharmaceutical composition comprising one or more pharmaceutically acceptable excipients, carriers, and diluents. Pharmaceutically acceptable excipients, carriers and diluents can be included to increase shelf-life, stability, or the administrability of the antibody. Such compounds include salts, pH buffers, detergents, anti-coagulants, and preservatives. In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the conjugates of the current disclosure can be shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the conjugates described herein in a suitable container and one or more additional components selected from: instructions for use; a diluent, an excipient, a carrier, and a device for administration.

### Methods of Use

Also provided herein is a method of treating a disease or disorder in a subject in need thereof. In some embodiments, the method comprises administering to the subject an effective amount of a conjugate disclosed herein. In some embodiments, the method comprises administering to the subject an effective amount of a conjugate of Formula (I). In some embodiments, the method comprises administering to the subject a pharmaceutical composition disclosed herein (i.e., comprising an effective amount of a conjugate provided herein). In some embodiments, the disease or disorder is a cancer or tumor.

In some embodiments, provided herein is a method of treating cancer or tumor in a subject in need thereof, the method comprising administering to the subject an effective amount of the conjugate of Formula (I), or a stereoisomer thereof, or a pharmaceutically acceptable salt thereof. In some embodiments, provided herein is a method of treating cancer or tumor in a subject in need thereof, the method comprising administering to the subject a pharmaceutical composition comprising an effective amount of the conjugate of any one of Formula (I), or a stereoisomer thereof, or a pharmaceutically acceptable salt thereof.

Alternatively, another aspect of the disclosure is a conjugate of Formula (I), or a stereoisomer thereof, or a pharmaceutically acceptable salt thereof for use in a method of treating a cancer or tumor (or for use in the treatment of cancer or tumor). In some embodiments, provided herein is a conjugate of any one of Formula (I), or a stereoisomer thereof, or a pharmaceutically acceptable salt thereof for use in the manufacture of a medicament for treating cancer or tumor.

In some embodiments, the subject in need thereof is a human or non-human primate. In some embodiments, the subject is a human. In some embodiments, the cancer or tumor is any one of the cancers disclosed herein. In some embodiments, the method comprises treating a disease or condition described herein. In some embodiments, disease or condition comprises solid tumor. In some embodiments, the disease or condition comprises cancer. In some embodiments, the cancer comprises non-small cell lung cancer (NSCLC); ovarian cancer; breast cancer; gastrointestinal cancer; prostate cancer; head and neck cancer; endometrial cancer; cervical cancer; or a combination thereof

As stated above, one aspect of the invention is a method of treating cancer using a conjugate disclosed herein. As used herein, the term “cancer” generally refers to all types of cancer, neoplasm or malignant tumors found in mammals (e.g., humans). In some embodiments, cancer includes non-small cell lung cancer (NSCLC); ovarian cancer; breast cancer; gastrointestinal cancer; prostate cancer; head and neck cancer; endometrial cancer; cervical cancer; or a combination thereof.

### Chemical Compositions

In another aspect, provided herein are chemical compositions comprising a drug (D) having the structure of Formula (Ia), and a polypeptide. In some embodiments, the drug having the structure of Formula (Ia) is a topoisomerase inhibitor. In some embodiments, the drug is Formula (Ia), and the polypeptide is an antibody or antigen-binding fragment thereof. In some embodiments, the chemical composition further comprises a reaction medium (e.g., a solvent). In some embodiments, the drug is covalently bonded to the polypeptide via a linker. In some embodiments, the linker comprises at least one amino acid. In some embodiments, the linker comprises at least one amino acid that is an L-amino acid. In some embodiments, the linker comprises at least one amino acid that is an D-amino acid. In some embodiments, the linker comprises at least two amino acids. In some embodiments, the linker comprises at least two amino acids, where one or more of the at least two amino acids comprise the L-amino acid. In some embodiments, the linker comprises at least two amino acids, where one or more of the at least two amino acids comprise the D-amino acid. In some embodiments, the linker comprises at least three amino acids, where one or more of the at least three amino acids comprise the L-amino acid. In some embodiments, the linker comprises at least three amino acids, where one or more of the at least three amino acids comprise the D-amino acid. In some embodiments, the linker comprises two or more amino acids, where the two or more amino acids comprise at least one L-amino acid, at least one D-amino acid, or a combination thereof.

In some embodiments, the linker comprises one, two, or three amino acids. In some embodiments, the linker comprising the one, two, or three amino acids, where the one, two, or three amino acids comprise at least one L-amino acid, at least one D-amino acid, or a combination thereof. In some embodiments, the linker comprises three amino acids, where the three amino acids comprise at least one L-amino acid, at least one D-amino acid. In some embodiments, the linker comprises three amino acids, where the three amino acids comprise L-amino acids. In some embodiments, the linker comprises three amino acids, where the three amino acids comprise D-amino acids. In some embodiments, the linker comprises three amino acids, where the three amino acids comprise the same amino acids. In some embodiments, the linker comprises three amino acids, where the three amino acids comprise two or more different amino acids. In some embodiments, the linker comprises three amino acids, where the three amino acids comprise three alanines (tri-alanine). In some embodiments, the linker comprises three alanines (tri-alanine), where the three alanines comprise at least one L-alanine or at least one D-alanine. In some embodiments, the linker comprises three alanines (tri-alanine), where the three alanines comprise three L-alanines. In some embodiments, the linker comprises three alanines (tri-alanine), where the three alanines comprise three D-alanines.

In some embodiments, D comprises the structure of Formula (Ia):

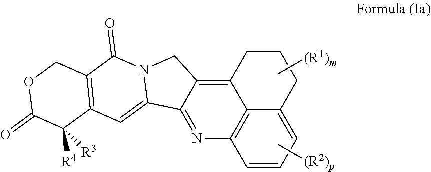

or a stereoisomer thereof, or a pharmaceutically acceptable salt thereof, wherein:
each R¹, R², R³, and R⁴ is independently H, D, F, Cl, Br, I, OH, C₁-C₆ alkyl, C₁-C₆ heteroalkyl, C₁₋₄ alkoxy, C₁₋₄ alkylamino, 5- to 6-membered heteroaryl, 3- to 10-membered heterocycloalkyl, each of which is optionally substituted with 1, 2, or 3 R^{a};
each R^{a} is independently H, D, F, Cl, Br, I, OH, C₁₋₃ alkyl, C₁₋₃ alkoxy, C₁₋₃ alkylamino, CHO, C(O)CH₃, wherein each C₁₋₃ alkyl, C₁₋₃ alkoxy, and C₁₋₃ alkylamino, is optionally substituted with 1, 2 or 3 R;
each R is independently D, F, Cl, Br, I, OH, CH₃, CF₃, NH₂, OCH₃, CHO, C(O)CH₃, or OCF₃; and
m, and p, and are each independently 0, 1, 2, or 3;
wherein each “3- to 6-membered heterocycloalkyl”, “3- to 10-membered heterocycloalkyl”, and “5- or 6-membered heteroaryl” each independently contains 1 or 2 heteroatoms or heteroatom groups independently selected from —NH—, —O—, —S—, and N.

In some embodiments of Formula (Ia), m is 1. In some embodiments of Formula (Ia), p is 2. In some embodiments of Formula (Ia), m is 1 and p is 2. In some embodiments of Formula (Ia), R² is F. In some embodiments of Formula (Ia), R² is CH₃. In some embodiments of Formula (Ia), R² is CH₃. In some embodiments of Formula (Ia), R¹ is NH₂. In some embodiments of Formula (Ia), R⁴ is CH₂CH₃. In some embodiments of Formula (Ia), R³ is OH. In some embodiments of Formula (Ia), X is attached to L via R¹. In some embodiments of Formula (Ia), D comprises the structure:

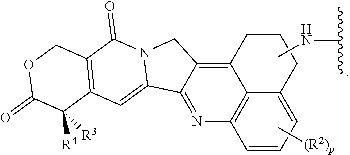

In some embodiments of Formula (Ia), D comprises the structure:

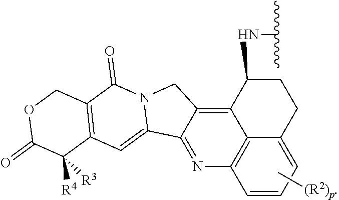

In some embodiments of Formula (Ia), D comprises the structure:

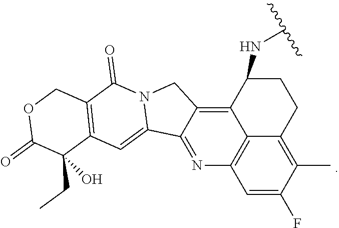 [SMILES: *N[C@H]1CCc2c(C)c(F)cc3nc4c(c1c23)Cn1c-4cc2c(c1=O)COC(=O)[C@]2(O)CC]

In some embodiments, each L independently comprises the structure of Formula (Ib):

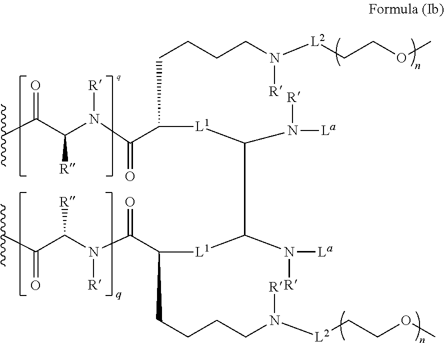

or a stereoisomer thereof, or a pharmaceutically acceptable salt thereof, wherein:
each R″ is independently hydrogen or C₁₋₆ alkyl, wherein the C₁₋₆ alkyl is optionally substituted with phenyl, —C(═O)NR′₂, —C(═O)OR′, —OR′, —NR′₂, —SR′, —NR′C(═O)NR′₂, or —NR′C(═NH)NR′₂, and
each R′ is independently —H or —CH₃;
each n is 1-15;
each q is 1-3;
each L¹ is —NR′C(O)(CH₂)₂₋₄—NR′C(O)—, —C(O)NR′(CH₂)₂₋₄—C(O)NR′—, —(CH₂)₁₋₆—, —OC(O)(CH₂)₂₋₄—NR′C(O)—, or —NR′C(O)(CH₂)₂₋₄—OC(O)—;
each L² is —C(O)(CH₂)₁₋₃O—, —C(O)(CH₂)₁₋₃NR′—, —C(O)NR′(CH₂)₁₋₃O—, or —(CH₂)₁₋₃—;
and
each L^{a} is independently

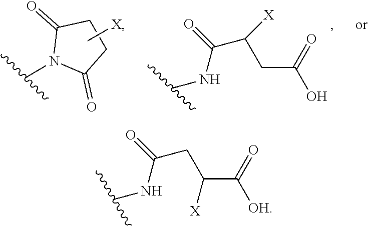

In some embodiments of Formula (Ib), q is 3. In some embodiments of Formula (Ib), n is 8-10. In some embodiments of Formula (Ib), n is 9. In some embodiments of Formula (Ib), R′ is H. In some embodiments of Formula (Ib), L¹ is —NR′C(O)(CH₂)₂₋₄—NR′C(O)—. In some embodiments of Formula (Ib), L¹ is —NR′C(O)(CH₂)₃—NR′C(O)—. In some embodiments of Formula (Ib), L² is —C(O)(CH₂)₁₋₃O—. In some embodiments of Formula (Ib), L² is —C(O)(CH₂)O—.

In some embodiments, each L independently comprises the structure of Formula (Ic):

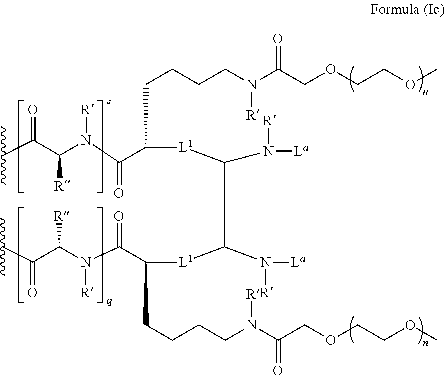

or a stereoisomer thereof, or a pharmaceutically acceptable salt thereof, wherein:
each R″ is independently hydrogen or C₁₋₆ alkyl, wherein the C₁₋₆ alkyl is optionally substituted with phenyl, —C(═O)NR′₂, —C(═O)OR′, —OR′, —NR′₂, —SR′, —NR′C(═O)NR′₂, or —NR′C(═NH)NR′₂; and
each R′ is independently —H or —CH₃;
each n is 1-15;
each q is 1-3;
each L¹ is —NR′C(O)(CH₂)₂₋₄—NR′C(O)—, —C(O)NR′(CH₂)₂₋₄—C(O)NR′—, —(CH₂)₁₋₆—, —OC(O)(CH₂)₂₋₄—NR′C(O)—, or —NR′C(O)(CH₂)₂₋₄—OC(O)—;
and
each L^{a} is independently

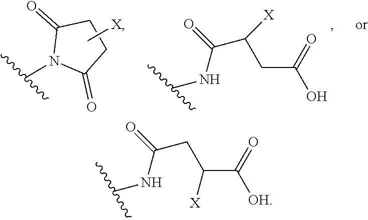

In some embodiments of Formula (Ic), q is 3. In some embodiments of Formula (Ic), n is 8-10. In some embodiments of Formula (Ic), n is 9. In some embodiments of Formula (Ic), R′ is H. In some embodiments of Formula (Ic), L¹ is —NR′C(O)(CH₂)₂₋₄—NR′C(O)—. In some embodiments of Formula (Ic), L¹ is —NR′C(O)(CH₂)₃—NR′C(O)—.

In some embodiments, each L independently comprises the structure of Formula (Id):

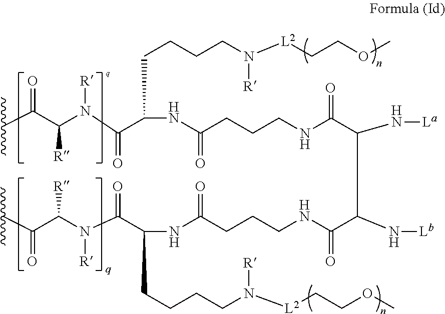

or a stereoisomer thereof, or a pharmaceutically acceptable salt thereof, wherein:
each R″ is independently hydrogen or C₁₋₆ alkyl, wherein the C₁₋₆ alkyl is optionally substituted with phenyl, —C(═O)NR′₂, —C(═O)OR′, —OR′, —NR′₂, —SR′, —NR′C(═O)NR′₂, or —NR′C(═NH)NR′₂; and
each R′ is independently —H or —CH₃;
each n is 1-15;
each q is 1-3;
each L² is —C(O)(CH₂)₁₋₃O—, —C(O)(CH₂)₁₋₃NR′—, —C(O)NR′(CH₂)₁₋₃O—, or —(CH₂)₁₋₃—;
and
each L is independently

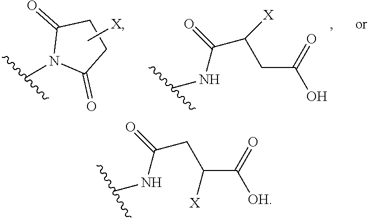

In some embodiments of Formula (Ic), q is 3. In some embodiments of Formula (Ic), n is 8-10. In some embodiments of Formula (Ic), n is 9. In some embodiments of Formula (Ic), R′ is H. In some embodiments of Formula (Ic), L² is —C(O)(CH₂)₁₋₃O—. In some embodiments of Formula (Ic), L² is —C(O)(CH₂)O—.

In some embodiments, each L independently comprises the structure of Formula (Ie):

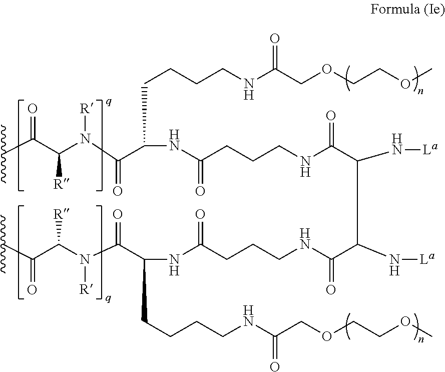

or a stereoisomer thereof, or a pharmaceutically acceptable salt thereof, wherein:
each R″ is independently hydrogen or C₁₋₆ alkyl, wherein the C₁₋₆ alkyl is optionally substituted with phenyl, —C(═O)NR′₂, —C(═O)OR′, —OR′, —NR′₂, —SR′, —NR′C(═O)NR′₂, or —NR′C(═NH)NR′₂; and
each R′ is independently —H or —CH₃;
each n is 1-15;
each q is 1-3; and
each L^{a} is independently

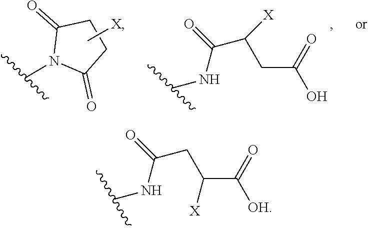

In some embodiments of Formulas (Ib-Ie),

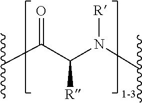

comprises

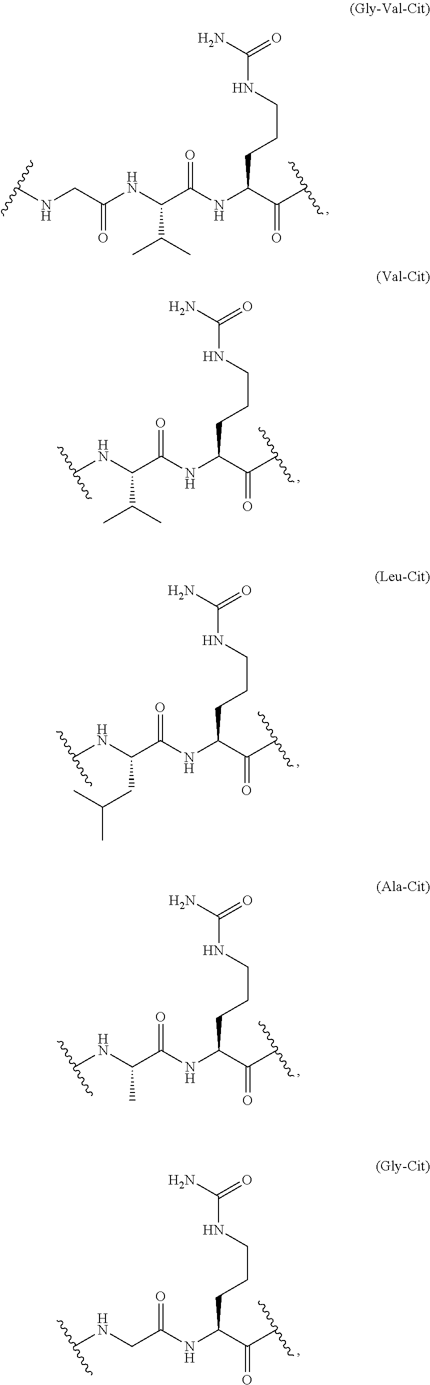 [SMILES: *NCC(=O)N[C@H](C(=O)N[C@@H](CCCNC(N)=O)C(*)=O)C(C)C ; *N[C@H](C(=O)N[C@@H](CCCNC(N)=O)C(*)=O)C(C)C ; *N[C@@H](CC(C)C)C(=O)N[C@@H](CCCNC(N)=O)C(*)=O ; *N[C@@H](C)C(=O)N[C@@H](CCCNC(N)=O)C(*)=O ; *NCC(=O)N[C@@H](CCCNC(N)=O)C(*)=O]

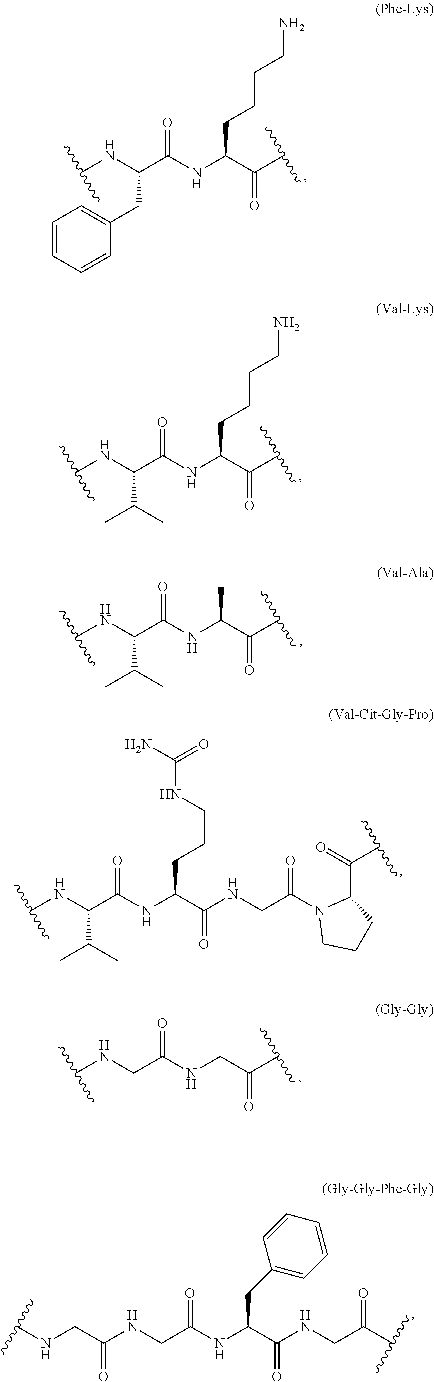 [SMILES: *N[C@@H](Cc1ccccc1)C(=O)N[C@@H](CCCCN)C(*)=O ; *N[C@H](C(=O)N[C@@H](CCCCN)C(*)=O)C(C)C ; *N[C@H](C(=O)N[C@@H](C)C(*)=O)C(C)C ; *N[C@H](C(=O)N[C@@H](CCCNC(N)=O)C(=O)NCC(=O)N1CCC[C@H]1C(*)=O)C(C)C ; *NCC(=O)NCC(*)=O ; *NCC(=O)NCC(=O)N[C@@H](Cc1ccccc1)C(=O)NCC(*)=O]

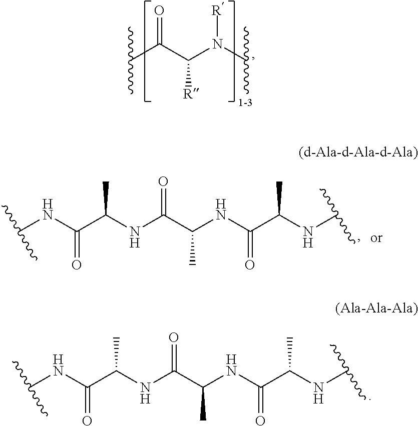

In some embodiments, each L independently comprises the structures:

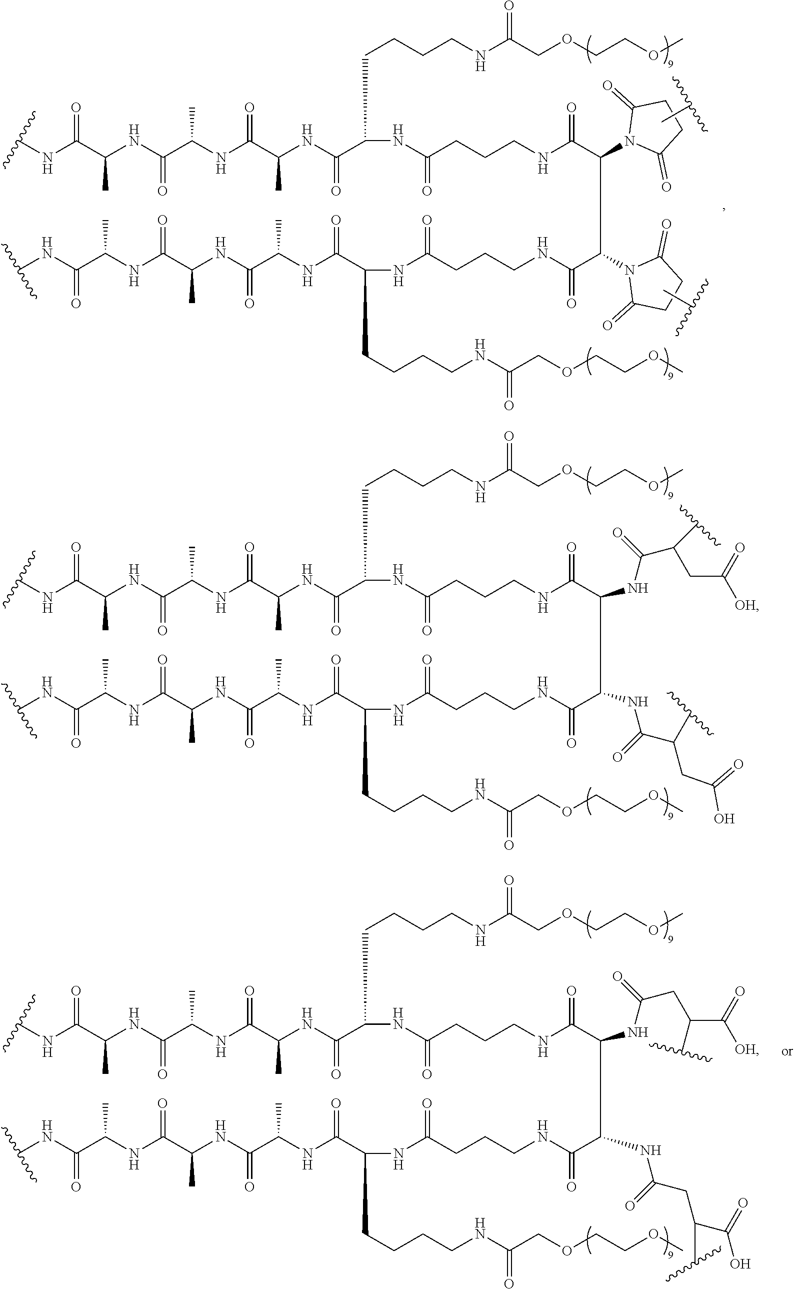

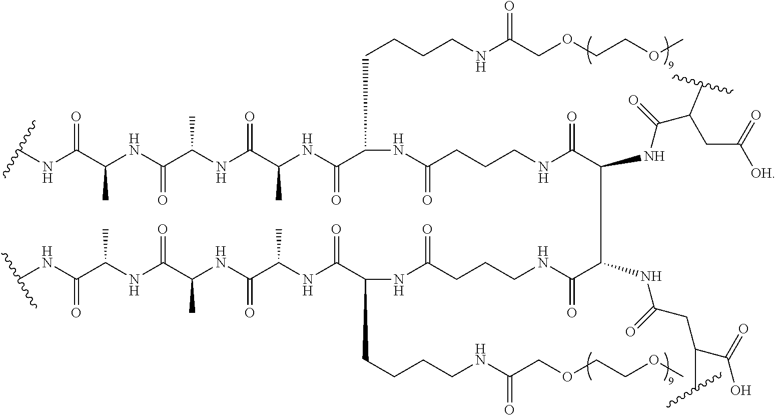

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical Formula, all combinations and sub-combinations of ranges and specific embodiments therein are intended to be included. As used herein the term “about” refers to an amount that is near the stated amount by 10% or less.

Unless defined otherwise, all technical and scientific terms used herein generally have the same meaning as commonly understood by one of ordinary skill in the art to which this disclosure belongs. However, to facilitate understanding of the disclosure set forth herein, a number of terms are defined below.

As used in the specification and appended claims, unless specified to the contrary, the following terms have the meaning indicated below.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an polypeptide of interest and the products screened for a desired activity, for example, retained/improved antigen binding, decreased immunogenicity, or improved ADCC or CDC.

TABLE 1
Amino acid residues

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

In some embodiments, the polypeptide described herein comprises at least one amino acid substitution. In some embodiments, the amino acid substitution can include a conservative substitution, where an amino acid is substituted by another amino acid in the same class (e.g., aliphatic class, hydroxyl or sulfur/selenium-containing class, aromatic class, basic class, or acidic and their amides class). In some embodiments, the aliphatic class comprises glycine (G), alanine (A), valine (V), leucine (L), or isoleucine (I). In some embodiments, the hydroxyl or sulfur/selenium-containing class comprises serine (S), cysteine (C), selenocysteine (U), threonine (T), or methionine (M). In some embodiments, the aromatic class comprises phenylalanine, (F), tyrosine (Y), or tryptophan (W). In some embodiments, the basic class comprises histidine (H), lysine (K), or arginine (R). In some embodiments, the hydroxyl or sulfur/selenium-containing class comprises aspartate (D), glutamate (E), asparagine (N), or glutamine (Q).

As used herein, C₁-Cₓ includes C₁-C₂, C₁-C₃ . . . C₁-Cₓ. C₁-Cₓ refers to the number of carbon atoms that make up the moiety to which it designates (excluding optional substituents), and is inclusive of both C₁ and Cₓ, where x is an integer value.

“Administering,” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering,” when used in conjunction with a composition described herein, can include, but is not limited to, providing a composition into or onto the target tissue; providing a composition systemically to a patient by, e.g., oral administration whereby the therapeutic reaches the target tissue or cells. “Administering” a composition may be accomplished by injection (parenteral, intravenous, subcutaneous, intraperitoneal, intramuscular, intravascular, intrathecal, intravitreal, infusion, or local), topical, oral, or nasal administration, or by other methods alone or in combination with other known techniques.

As used herein, the term “alkyl,” generally refers to a branched or straight chain aliphatic hydrocarbon group. As used in the present context, an alkyl group generally comprises 1 to 100 carbon atoms, though a select range may be otherwise specified (i.e., an alkyl group comprising 1 to 12 carbon atoms may be denoted as C₁-C₁₂ alkyl. Whenever it appears herein, a numerical range such as “1 to 12” refers to each integer in the given range; e.g., “1 to 12 carbon atoms” means that the alkyl group consist of 1 carbon atom, 2 carbon atoms, 3 carbon atoms, etc., up to and including 12 carbon atoms, although the present definition also covers the occurrence of the term “alkyl” where no numerical range is designated. In some embodiments, an alkyl is a C₁-C₆ alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a branched or straight chain divalent alkyl radical. Any of the above mentioned monovalent alkyl groups may be an alkylene by abstraction of a second hydrogen atom from the alkyl. In some embodiments, an alkylene is a C₁-C₆ alkylene. In other embodiments, an alkylene is a C₁-C₄ alkylene. Typical alkylene groups include, but not limited to, —CH₂—, —CH₂CH₂—, —CH₂CH₂CH₂—, —CH₂CH₂CH₂CH₂—, and the like. In some embodiments, an alkylene is —CH₂—. In some instances, an alkylene group is substituted by one or more substituents, in which case the one or more substituents replaces the equivalent number of hydrogen atoms to complete the valency and saturation of the alkylene group.

An “alkoxy” group refers to a —O(alkyl) group, where alkyl is as defined herein. Examples of alkoxy groups include —OCH₃, —OCH₂CH₃, —OCH₂CH₂CH₃, —OC(CH₃)₃, and the like.

An “aminoalkyl” refers to an alkyl in which one hydrogen atom is replaced by an amino. In some embodiments, aminoalkyl is a C₁-C₄aminoalkyl. Typical aminoalkyl groups include, but not limited to, —CH₂NH₂, —CH₂CH₂NH₂, —CH₂CH₂CH₂NH₂, —CH₂CH₂CH₂CH₂NH₂, —C(CH₃)₂NH₂, and the like.

The term “antigen” as provided herein refers to molecules capable of binding to the antibody binding domain provided herein. An “antigen binding domain” as provided herein is a region of an antibody that binds to an antigen (epitope). As described above, the antigen binding domain may include one constant and one variable domain of each of the heavy and the light chain (VL, VH, CL and CH1, respectively). In some embodiments, the antigen binding domain includes a light chain variable domain and a heavy chain variable domain. In embodiments, the antigen binding domain includes light chain variable domain and does not include a heavy chain variable domain and/or a heavy chain constant domain. The paratope or antigen-binding site is formed on the N-terminus of the antigen binding domain. The two variable domains of an antigen binding domain may bind the epitope of an antigen. Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH₁ by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries. Preferred in the context of the present invention are antigens associated with tumors or cancers, and agents that bind tumor- or cancer-associated antigens. In some instances an antibody binding domain binds to about 1, 2, 3, 4, 5, 6, 7, 8, 9, 10, 11, 12, 13, 14, or about 15 amino acid residues of the antigen. In some instances an antibody binding domain binds to at least 1, 2, 3, 4, 5, 6, 7, 8, 9, 10, 11, 12, 13, 14, or at least 15 amino acid residues of the antigen. In some instances an antibody binding domain binds to 1-5, 1-10, 1-12, 1-15, 2-10, 3-10, 4-10, 5-8, 5-10, 5-12, 6-10, 6-9, or 7-8 amino acid residues of the antigen.

As used herein, the term “antigen binding fragment” generally refers to a polypeptide capable of binding to an antigen and including one or more structural domains of an antibody or fragment thereof. Nonlimiting examples of antibody variants include single-domain antibodies (nanobodies), affibodies (polypeptides smaller than monoclonal antibodies (e.g., about 6 kDA) and capable of binding antigens with high affinity and imitating monoclonal antibodies, monospecific Fab2, bispecific Fab2, trispecific Fab3, monovalent IgGs, scFv, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody.

As used herein, the term “amino acid” or “amino acid residue” generally refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, g-carboxyglutamate, and O-phosphoserine.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, g-carboxyglutamate, and O-phosphoserine. Some amino acid side-chains contain reactive groups such as thiols and amines that may react with a reactive group such as maleimides or NHS esters, respectively. An amino acid side-chain specifically refers to the portion extending from the alpha carbon of the polypeptide backbone.

As used herein, the term “aryl,” generally refers to a monocyclic aromatic group and/or polycyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl and naphthyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl. The term “arylene” refers to a bivalent aryl group, which is substituted at two positions. The term “aryltriene” refers to a trivalent aryl group, which is substituted at three positions.

The term “cancer,” as used herein, refers to the presence of cells possessing characteristics typical of cancer-causing cells, such as uncontrolled proliferation, immortality, metastatic potential, rapid growth and proliferation rate, and/or certain morphological features. Often, cancer cells can be in the form of a tumor or mass, but such cells may exist alone within a subject, or may circulate in the blood stream as independent cells, such as leukemic or lymphoma cells. The term “cancer” includes all types of cancers and cancer metastases, including hematological cancers, solid tumors, sarcomas, carcinomas and other solid and non-solid tumor cancers.

As used herein, the terms “conjugate,” “antibody-drug conjugate” and “ADC” may be used interchangeably to generally refer to a compound comprising a linker that conjoins a drug (e.g., a topoisomerase inhibitor) to a polypeptide, which in some embodiments, is an antibody or antigen-binding fragment. As used herein, conjugates and compounds are generally differentiable in that a conjugate comprises a polypeptide (“X”), whereas a compound comprises a reactive group (“Y”).

As used herein, the term “cycloalkyl,” generally refers to a non-aromatic, monocyclic or polycyclic ring system. In some embodiments, the cycloalkyl is a mono- or bi-cyclic saturated carbocyclic group containing from 3 to 10 ring members, which may include fused, bridged or spiro ring systems. Examples of cycloalkyl include cyclopropyl, cyclobutyl, cyclopentyl, and cyclohexyl. The term “cycloalkylene” refers to a bivalent cycloalkyl group, which is substituted at two positions. By extension, the term “cycloalkyltriene” refers to a trivalent cycloalkyl group.

As used herein, the term “heteroalkyl” generally refers to a monovalent straight or branched alkyl chain (a saturated hydrocarbon chain), wherein one or more methylene units is replaced with a group selected from: —C(═O)—, —NH—, —NCH₃—, —O—, or —S—, or any combination thereof (i.e., —C(═O)NH—, —C(═O)O—, —NCH₃C(═O)NH—, etc.). The term “heteroalkylene” refers to a bivalent heteroalkyl group. Each heteroalkyl or heteroalkylene may be substituted at one or more carbon atoms with one or more R″ group, as defined herein. A heteroalkyl or heteroalkylene may comprise one or more polymeric units, e.g., —CH₂CH₂O—, —CH₂CH₂NH—, —CH₂CH₂NCH₃—, —CR″₂C(═O)NH—, —CHR″C(═O)NH—, —C′R″C(═O)NCH₃—, —CH₂C(═O)NCH₃—.

As used herein, the term “heteroaryl” generally refers to a monovalent, monocyclic or polycyclic ring structure that contains at least one ring heteroatom selected from N, O, or S (i.e., a heterocycle), and at least one fully aromatic ring: i.e., they contain 4n+2 pi electrons within the monocyclic or polycyclic ring system. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, imidazolyl, thiazolyl, indolyl, quinolinyl, tetrahydroquinolinyl, benzoimidazolyl, benzothiazolyl, benzothiophenyl, benzodioxanyl, benzopyranyl, benzoxazolyl, chromanyl. In some embodiments, the heteroaryl any mono- or bi-cyclic group composed of from 5 to 10 ring members, having at least one aromatic moiety and containing from 1 to 4 hetero atoms selected from oxygen, sulfur and nitrogen (including quaternary nitrogens). The term “heteroarylene” refers to a bivalent heteroaryl group, which is substituted at two positions. By extension, the term “heteroaryltriene” refers to a trivalent heteroaryl group, which is substituted at three positions.

As used herein, the term “heterocycloalkyl,” generally refers to a monovalent monocyclic, bicyclic or polycyclic ring system, which may be bridged, fused or spirocyclic, wherein at least one ring is non-aromatic and contains one, two, three, or four ring heteroatoms independently selected from the group consisting of nitrogen, oxygen, and sulfur. Examples of heterocycloalkyl groups include pyrrolidinyl, piperidinyl, piperazinyl, morpholinyl. Unless otherwise specified, a heterocycloalkyl group may have between 3 and 30 ring atoms. In some embodiments, a heterocycloalkyl group is a 5- or 6-membered (e.g., monocyclic) heterocycloalkyl group.

As used herein, the term “inhibit” or “inhibition” or “inhibiting,” generally means to reduce biological activity by a measurable amount, and can include but does not require complete inhibition. In some embodiments, “inhibition” means to reduce the expression and/or activity of PTK7 and/or one or more upstream modulators or downstream targets thereof. The term “inhibit” is meant to encompass various forms of inhibition, including binding and stabilizing protein-protein interactions that cause a decrease in activity (i.e., a molecular glue).

The terms “polypeptide,” “peptide” and “protein” are used interchangeably herein to refer to a polymer of amino acid residues, wherein the polymer may in embodiments be conjugated to a moiety that does not consist of amino acids. A polypeptide or peptide comprises at least one amino acid, which is generally bonded at both the N- and C-termini to an adjacent amino acid, thereby forming the peptide or polypeptide. In sufficiently large, functional assemblies, polypeptides are referred to as proteins. In some embodiments, a peptide or polypeptide refers to an antibody, or to an antigen-binding fragment of an antibody. In some embodiments, polypeptide or peptide refers to a cysteine or a lysine amino acid, bonded on either end to an adjacent amino acid, thereby forming said peptide or polypeptide (or antibody or antigen-binding fragment, as the case may be).

As used herein, a “reactive group” or “reactive moiety” or “moiety capable of reacting with a thiol or an amine side-chain of a peptide or polypeptide” may be used interchangeably. In some embodiments, the reactive group is an electrophilic group. In some embodiments, the reactive group forms a bond to an amino acid side-chain or polypeptide after reacting with (conjugating to) a thiol of a cysteine side-chain or amine of a lysine side-chain of the targeting peptide or polypeptide. In some embodiments, the reactive groups comprises a succinimide or maleimide group. In some embodiments, the reactive groups comprises a maleimide group. In some embodiments, the maleimide group is also referred to as a maleimide spacer. In some embodiments, the maleimide group further comprises a caproic acid, forming maleimidocaproyl (mc). In some embodiments, the linker comprises maleimidocaproyl (mc). In some embodiments, a maleimide conjugate (e.g., thiosuccinimide) is further hydrolyzed to generate a ring-opened product (e.g., succinamic acid thioether). In some instances a linker described herein comprises one or more reactive groups. In some instances a linker described herein comprises one or more reactive groups configured to react with a drug and/or polypeptide. In some instances a linker described herein comprises one, two, three, four, or more than four reactive groups.

The term “subject” refers to an animal, including, but not limited to, a primate (e.g., human, chimpanzee, orangutan, rhesus macaque, etc.), a dog, a cat, a rabbit, a rat, or a mouse. The terms “subject” and “patient” and “individual” are used interchangeably herein in reference, for example, to a mammalian subject, such as a human subject.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to therapeutic treatment, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

As used herein, the term “tumor” generally refers to any mass of tissue that results from excessive cell growth or proliferation, either benign or malignant, including precancerous lesions and cancerous tumors.

### EXAMPLES

### Example 1. Synthesis of a PTK7 ADC (ADC-A)

A linker payload having the structure:

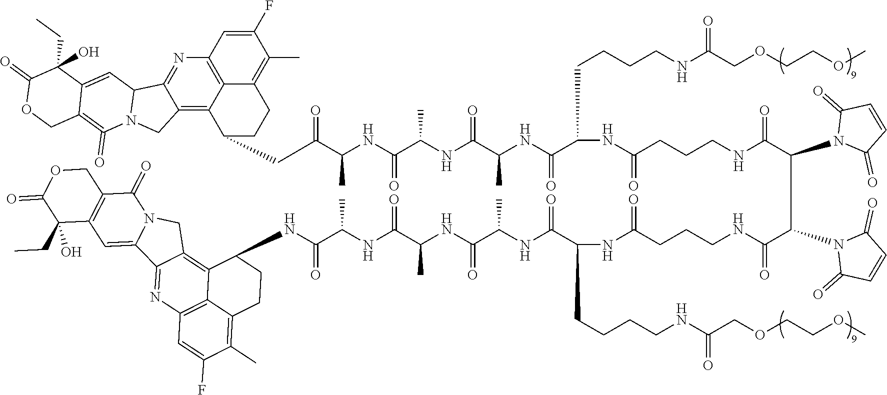

was synthesized and conjugated to a PTK7 antibody using the general methods of WO2023/078273, which is incorporated by reference in its entirety, to generate a compound of ADC-A having: a heavy chain sequence of SEQ ID NO: 1; a light chain sequence of SEQ ID NO: 2; and a drug to antibody ratio (DAR) of six.

### Example 2. Therapeutic Efficacy of PTK7 Antibody Conjugate (ADC-A)

The binding and therapeutic efficacy of the PTK7 antibody and the PTK7 antibody conjugated to a linker payload (ADC-A) was examined in multiple cell line or xenograft models.

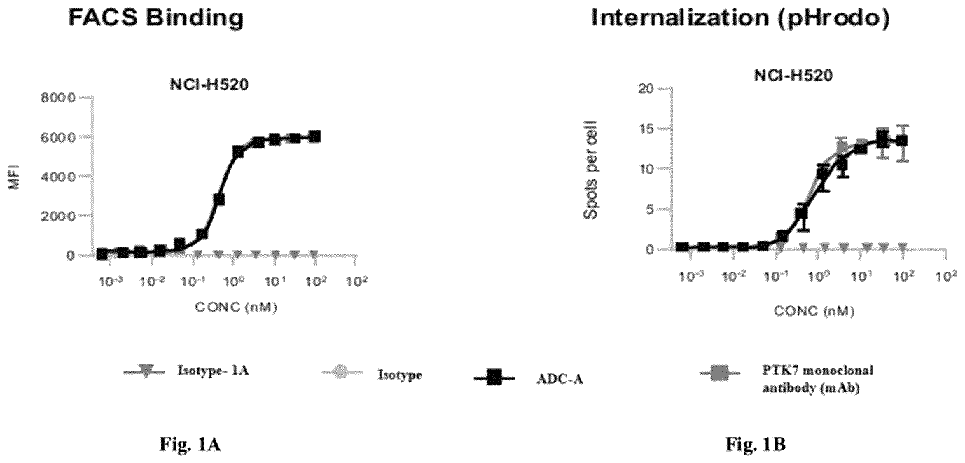

FIG. 1A illustrates comparable antigen (PTK7) binding exhibited by the PTK7 antibody and the PTK7 antibody conjugate (ADC-A). FIG. 1B illustrates comparable internalization exhibited by the PTK7 antibody and the ADC-A. PTK7 antibody ADC-A targeted PTK7 Ig domain, where the PTK7 antibody was IgG1 class antibody and exhibited by a binding affinity (Kd) of 0.16 nM). The conjugate comprised a linker comprising a proteolytically cleavable tri-alanine. The conjugate comprised a linker-payload (e.g., a drug described herein), where the payload was a topoisomerase 1 inhibitor. The drug to antibody ratio (DAR) of the conjugate tested was six.

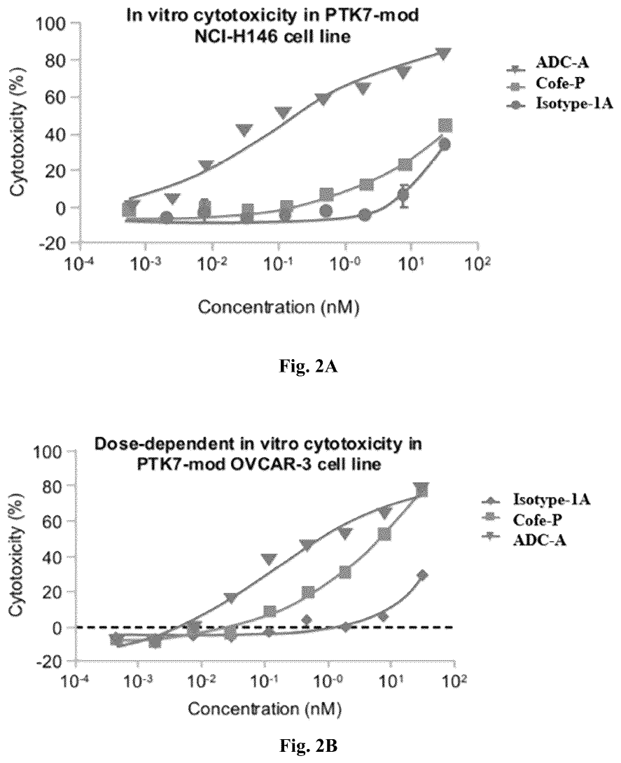

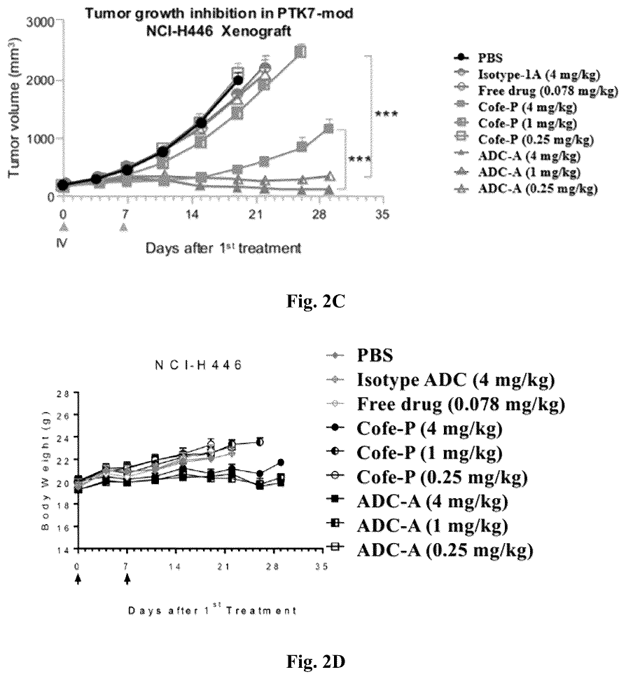

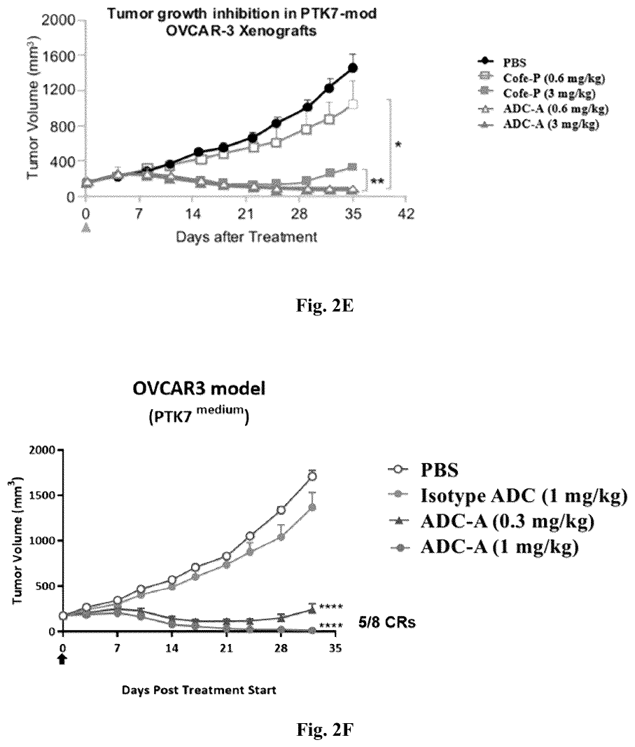

FIGS. 2A-2E illustrate improved therapeutic efficacy of ADC-A described herein compared to cofetuzumab (Cofe-P) and an isotype-1A ADC (control ADC without antigen binding property). FIG. 2A illustrates that ADC-A (inverted triangles) increased in vitro cytotoxicity in NCI-H146 lung carcinoma cells compared to cofetuzumab (Cofe-P, squares) or the isotype-1A ADC (circles). FIG. 2B illustrates that ADC-A (inverted triangles) increased dose-dependent in vitro cytotoxicity in OVCAR-3 adenocarcinoma of the ovary cells compared to cofetuzumab (Cofe-P, squares) or isotype-1A ADC control (circles). FIG. 2C illustrates that ADC-A (triangles) increased tumor growth inhibition in NCI-H446 small cell lung carcinoma xenograft model compared to cofetuzumab (Cofe-P, squares) or the isotype-1A ADC (circles). FIG. 2D illustrates body weights of the mice of the NCI-H446 xenograft model during the course of the ADC-A treatment. Table 5 illustrates percentage of tumor growth inhibition (TGI) and body weight change as measured on Day 19 post injection. FIG. 2E illustrates that ADC-A (triangles) increased tumor growth inhibition in OVCAR-3 xenograft model to cofetuzumab (Cofe-P, squares). FIG. 2F illustrates therapeutic efficacy against OVCAR3 ovarian cancer cell line-derived xenograft model.

TABLE 5
Tumor growth inhibition in NCI-H446 CDX model

| | | | Body weight change |
|---|---|---|---|
| ADCs | Dose (mg/kg) | TGI at Day 19 | at Day 19 |
| Isotype-CPT113 | 4 | 11.9% | 11.8% |
| CPT116 | 0.078 | −4.5% | 15.6% |
| ADC-A | 4 | 101.2%*** | 6.3% |
| ADC-A | 1 | 92.7%*** | 5.45% |
| ADC-A | 0.25 | 17.7% | 12.4% |
| Cofe-P | 4 | 83.8%*** | 3.4% |
| Cofe-P | 1 | 30.4%** | 12.3% |
| Cofe-P | 0.25 | −5.9% | 16.1% |

* represent p value < 0.05;
**represent p value < 0.01;
***represent p value < 0.005

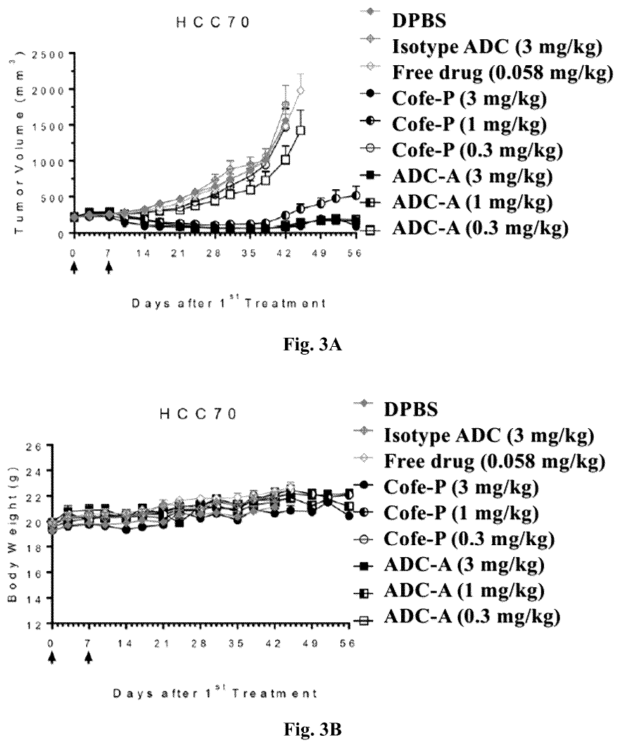

FIG. 3A illustrates therapeutic efficacy of tumor reduction of cell line-derived HCC70 triple-negative breast cancer (TNBC) xenograft (CDX) model of a conjugate described herein (ADC-A, squares) compared to tumor induced by a comparable antibody conjugate (cofetuzumab, “Cofe-P”, circles) and controls (diamonds). X-axis referenced days post injection with arrows indicating days of injection. Key: DPBS (solid diamonds); Isotype-1A ADC (Isotype-CPT-113, 3 mg/kg, half shaded diamonds); Free drug (CPT-116, 0.058 mg/kg, unshaded diamonds); Cofe-P (3 mg/kg, solid circles); Cofe-P (1 mg/kg, half shaded circles); Cofe-P (0.3 mg/kg, unshaded circles); ADC-A (3 mg/kg, solid squares); ADC-A (1 mg/kg, half shaded squares); ADC-A (0.3 mg/kg, unshaded squares). The x-axis is labeled days after first treatment from 0 to 5656 at 7 day intervals; the y-axis is labeled tumor volume (mm³) from 0 to 2500 at 500 mm³ intervals. FIG. 3B illustrates body weights of the mice of the HCC70 TNBC xenograft (CDX) model treated in FIG. 3A. Table 6 illustrates percentage of tumor growth inhibition (TGI) and body weight change as measured on Day 42 post injection.

TABLE 6
Tumor growth inhibition in HCC70 TNBC CDX model

| | | | Body weight change |
|---|---|---|---|
| ADCs | Dose (mg/kg) | TGI at Day 42 | at Day 42 |
| Isotype-CPT113 | 3 | −16.8% | 11.2% |
| CPT116 | 0.058 | 5.4% | 12.2% |
| ADC-A | 3 | 109.6%*** | 9.5% |
| ADC-A | 1 | 108.7%*** | 10.1% |
| ADC-A | 0.3 | 40.2% | 9.1% |
| Cofe-P | 3 | 110.1%*** | 6.9% |
| Cofe-P | 1 | 98.1%*** | 12.8% |
| Cofe-P | 0.3 | 6.3% | 10.3% |

* represent p value < 0.05;
** represent p value < 0.01;
***represent p value < 0.005

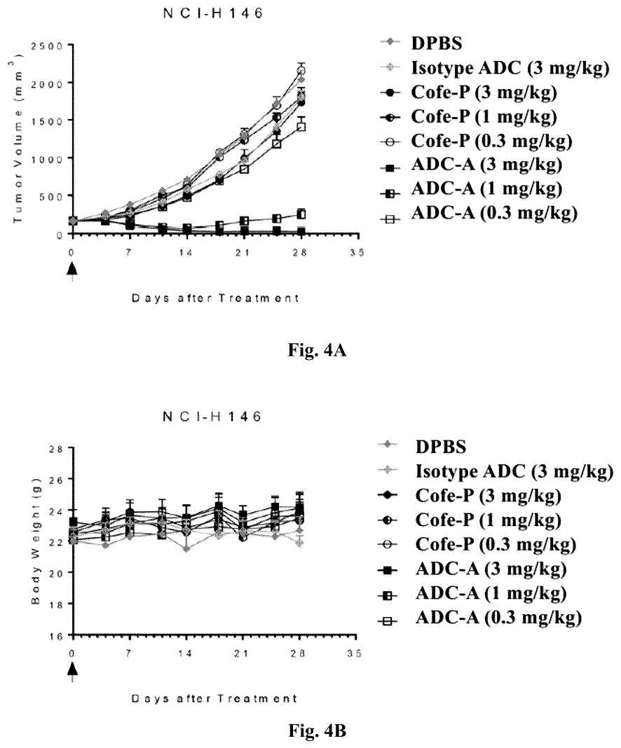

FIG. 4A illustrates therapeutic efficacy of tumor reduction of cell line-derived NCI-H146 small cell lung carcinoma (SCLC) xenograft (CDX) model of a conjugate described herein (ADC-A, squares) compared to tumor induced by a comparable antibody conjugate (cofetuzumab, “Cofe-P”, circles) and controls (diamonds). X-axis referenced days post injection with arrows indicating day of injection. Key: DPBS (solid diamonds); Isotype-1A ADC (Isotype-CPT-113, 3 mg/kg, half shaded diamonds); Cofe-P (3 mg/kg, solid circles); Cofe-P (1 mg/kg, half shaded circles); Cofe-P (0.3 mg/kg, unshaded circles); ADC-A (3 mg/kg, solid squares); ADC-A (1 mg/kg, half shaded squares); ADC-A (0.3 mg/kg, unshaded squares). The x-axis is labeled days after first treatment from 0 to 35 at 7 day intervals; the y-axis is labeled tumor volume (mm³) from 0 to 2500 at 500 mm³ intervals. FIG. 4B illustrates body weights of the mice of the NCI-H146 SCLC xenograft (CDX) model treated in FIG. 4A. Table 7 illustrates percentage of tumor growth inhibition (TGI) and body weight change as measured on Day 28 post injection.

TABLE 7
Tumor growth inhibition in NCI-H146 SCLC CDX model

| | | | Body weight change |
|---|---|---|---|
| ADCs | Dose (mg/kg) | TGI at Day 28 | at Day 28 |
| Isotype-CPT113 | 3 | 12.6% | −1.8% |
| ADC-A | 3 | 107.1%*** | 4.0% |
| ADC-A | 1 | 95.5%*** | 6.4% |
| ADC-A | 0.3 | 33.8%** | 5.1% |
| Cofe-P | 3 | 16.3% | 6.4% |
| Cofe-P | 1 | 12.2% | 2.4% |
| Cofe-P | 0.3 | −6.2% | 6.8% |

* represent p value < 0.05;
**represent p value < 0.01;
***represent p value < 0.005

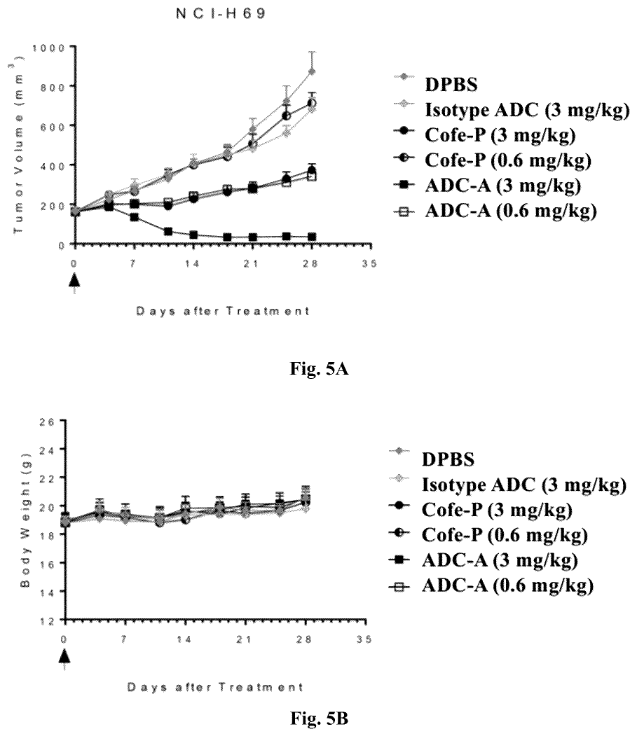

FIG. 5A illustrates therapeutic efficacy of tumor reduction of cell line-derived NCI-H69 SCLC cell line-derived xenograft (CDX) model of a conjugate described herein (ADC-A, squares) compared to tumor induced by a comparable antibody conjugate (cofetuzumab, “Cofe-P”, circles) and controls (diamonds). X-axis referenced days post injection with arrows indicating day of injection. Key: DPBS (solid diamonds); Isotype-1A ADC (Isotype-CPT-113, 3 mg/kg, half shaded diamonds); Cofe-P (3 mg/kg, solid circles); Cofe-P (0.6 mg/kg, half shaded circles); ADC-A (3 mg/kg, solid squares); and ADC-A (0.6 mg/kg, unshaded squares). The x-axis is labeled days after first treatment from 0 to 35 at 7 day intervals; the y-axis is labeled tumor volume (mm³) from 0 to 1000 at 200 mm³ intervals. FIG. 5B illustrates body weights of the mice of the NCI-H69 SCLC xenograft (CDX) model treated in FIG. 5A. Table 8 illustrates percentage of tumor growth inhibition (TGI) and body weight change as measured on Day 28 post injection.

TABLE 8
Tumor growth inhibition in NCI-H69 CDX model

| | | | Body weight change |
|---|---|---|---|
| ADCs | Dose (mg/kg) | TGI at Day 28 | at Day 28 |
| Isotype-CPT113 | 3 | 27.3% | 4.6% |
| ADC-A | 3 | 117.9%*** | 7.9% |
| ADC-A | 0.6 | 74.5%*** | 8.8% |
| Cofe-P | 3 | 70.2%** | 7.6% |
| Cofe-P | 0.6 | 22.4% | 7.6% |

* represent p value < 0.05;
**represent p value < 0.01;
***represent p value < 0.005

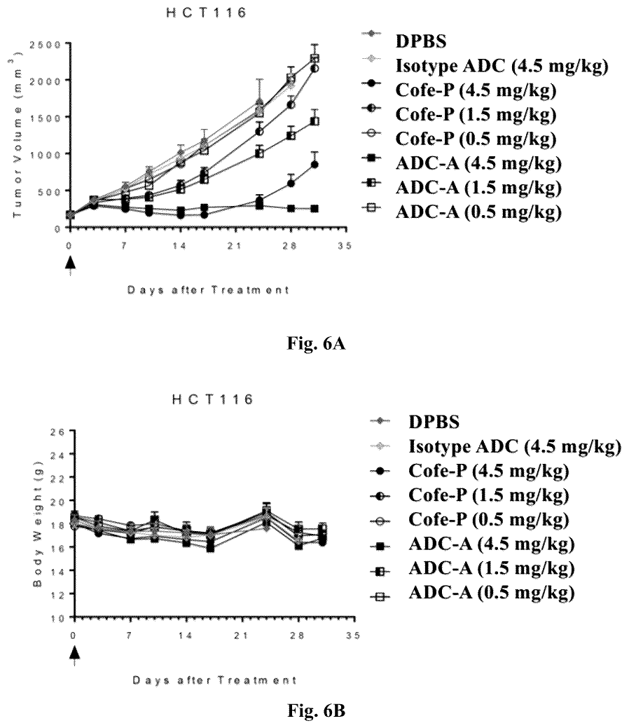

FIG. 6A illustrates therapeutic efficacy of tumor reduction of cell line-derived HCT-116 colorectal cancer cell (CRC) cell line-derived xenograft (CDX) model of a conjugate described herein (ADC-A, squares) compared to tumor induced by a comparable antibody conjugate (cofetuzumab, “Cofe-P”, circles) and controls (diamonds). X-axis referenced days post injection with arrows indicating day of injection. Key: DPBS (solid diamonds); Isotype-1A ADC (Isotype-CPT-113, 4.5 mg/kg, half shaded diamonds); Cofe-P (4.5 mg/kg, solid circles); Cofe-P (1.5 mg/kg, half shaded circles); Cofe-P (0.5 mg/kg, unshaded circles); ADC-A (4.5 mg/kg, solid squares); ADC-A (1.5 mg/kg, half shaded circles); and ADC-A (0.5 mg/kg, unshaded squares). The x-axis is labeled days after first treatment from 0 to 35 at 7 day intervals; the y-axis is labeled tumor volume (mm³) from 0 to 2500 at 500 mm³ intervals. FIG. 6B illustrates body weights of the mice of the HCT-116 CRC xenograft (CDX) model treated in FIG. 6A. Table 9 illustrates percentage of tumor growth inhibition (TGI) and body weight change as measured on Day 24 post injection.

TABLE 9
Tumor growth inhibition in HCT116 CDX model

| | | | Body weight change |
|---|---|---|---|
| ADCs | Dose (mg/kg) | TGI at Day 24 | at Day 24 |
| Isotype-CPT113 | 4.5 | 8.8% | 3.2% |
| ADC-A | 4.5 | 91.8%*** | 0.4% |
| ADC-A | 1.5 | 46.1%* | 3.0% |
| ADC-A | 0.5 | 10.5% | 0.9% |
| Cofe-P | 4.5 | 87.1%** | 2.8% |
| Cofe-P | 1.5 | 26.4% | 1.4% |
| Cofe-P | 0.5 | 7.9% | 6.7% |

*represent p value < 0.05;
**represent p value < 0.01;
***represent p value < 0.005

### Example 3. Synthesis and Evaluation of ADCs Targeting PTK7

Following the general methods of Examples 1 and 2, ADCs are generated using a combination of any sequences from Tables 2-4. In some embodiments, an ADC is generated comprising three heavy chain CDRs of Table 3 and thee light chain CDRs of Table 4.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and compositions within the scope of these claims and their equivalents be covered thereby.

TABLE 2
SEQUENCE TABLES

| | Amino acid sequence | Note |
|---|---|---|
| SEQ ID | QVQLVQSGAEVKKPGSSVKVSCKASGYTFTDYVIHWVRQAPGQGLEWMGYIN | Full |
| NO: 1 | PWSEGTKYNEKFKGRVTLTSDKSTSTAYMELSSLRSEDTAVYYCSRSGLPRYD | length |
| | AMDYWGQGTTVTVSSASTKGPSVFPLAPSSKSTSGGTAALGCLVKDYFPEPVT | heavy |
| | VSWNSGALTSGVHTFPAVLQSSGLYSLSSVVTVPSSSLGTQTYICNVNHKPSNT | chain |
| | KVDKRVEPKSCDKTHTCPPCPAPEAAGGPSVFLFPPKPKDTLMISRTPEVTCVV | |
| | VDVSHEDPEVKFNWYVDGVEVHNAKTKPREEQYNSTYRVVSVLTVLHQDWL | |
| | NGKEYKCKVSNKALPAPIEKTISKAKGQPREPQVYTLPPSREEMTKNQVSLTCL | |
| | VKGFYPSDIAVEWESNGQPENNYKTTPPVLDSDGSFFLYSKLTVDKSRWQQGN | |
| | VFSCSVMHEALHNHYTQKSLSLSPGK | |
| SEQ ID | DIVLTQSPASLAVSPGQRATITCRASQSVDYNALTYMNWLQQKPGQPPKLLIYA | Full |
| NO: 2 | ASTLESGVPARFSGSGSGTDFTLTINPVEAEDTANYYCQQSFRDPRTFGQGTKL | length |
| | EIKRTVAAPSVFIFPPSDEQLKSGTASVVCLLNNFYPREAKVQWKVDNALQSGN | light |
| | SQESVTEQDSKDSTYSLSSTLTLSKADYEKHKVYACEVTHQGLSSPVTKSFNRG | chain |
| | EC | |
| SEQ ID | QVQLVQSGAEVKKPGSSVKVSCKASGYTFTDYVIHWVRQAPGQGLEWMGYIN | Variable |
| NO: 3 | PWSEGTKYNEKFKGRVTLTSDKSTSTAYMELSSLRSEDTAVYYCSRSGLPRYD | heavy |
| | AMDYWGQGTTVTVSS | (VH) |
| | | chain |
| SEQ ID | DIVLTQSPASLAVSPGQRATITCRASQSVDYNALTYMNWLQQKPGQPPKLLIYA | Variable |
| NO: 4 | ASTLESGVPARFSGSGSGTDFTLTINPVEAEDTANYYCQQSFRDPRTFGQGTKL | light |
| | EIK | (VL) |
| | | chain |
| SEQ ID | GYTFTDYVIH | HCDR1 |
| NO: 5 | | |
| SEQ ID | YINPWSEGTKYNEKFKG | HCDR2 |
| NO: 6 | | |
| SEQ ID | SGLPRYDAMDY | HCDR3 |
| NO: 7 | | |
| SEQ ID | RASQSVDYNALTYMN | LCDR1 |
| NO: 8 | | |
| SEQ ID | AASTLES | LCDR2 |
| NO: 9 | | |
| SEQ ID | QQSFRDPRT | LCDR3 |
| NO: 10 | | |
| SEQ ID | IEDMPLFEPRVFTAGSEERVTCLPPKGLPEPSVWWEHAGVRLPTHGRVYQKGH | PTK7 |
| NO: 11 | ELVLANIAESDAGVYTCHAANLAGQRRQDVNITVA | antigen |

TABLE 3

| | Amino acid | | Amino acid | | Amino acid |
|---|---|---|---|---|---|
| SEQ ID | sequence of | SEQ ID | sequence of | SEQ ID | sequence of |
| NO | HCDR1 | NO | HCDR2 | NO | HCDR3 |
| SEQ ID | GFIFSNYAMH | SEQ ID | VISYDGNNKYYAD | SEQ ID | EVWSIDN |
| NO: 71 | | NO: 91 | SVKG | NO: 111 | |
| SEQ ID | GFTFSSYAFH | SEQ ID | VISYDGSIKYYADS | SEQ ID | TYYFDY |
| NO: 72 | | NO: 92 | VKG | NO: 112 | |
| SEQ ID | GFTFSTYLMY | SEQ ID | AIGSGGDTYYADS | SEQ ID | GLGY |
| NO: 73 | | NO: 93 | VKG | NO: 113 | |
| SEQ ID | GFTFSSYGMH | SEQ ID | VIWDDGSNKYYVD | SEQ ID | DDYYGSGSFNSYY |
| NO: 74 | | NO: 94 | SVKG | NO: 114 | GTDV |
| SEQ ID | GFSLSTSNMGVG | SEQ ID | HIWWDDDKYYSPS | SEQ ID | SNYGYAWFAY |
| NO: 75 | | NO: 95 | LKS | NO: 115 | |
| SEQ ID | GYTFTDYAVH | SEQ ID | VISTYNDYTYNNQ | SEQ ID | GNSYFYALDY |
| NO: 76 | | NO: 96 | DFKG | NO: 116 | |
| SEQ ID | GFDFSRYWMS | SEQ ID | DLNPDSSAINYVDS | SEQ ID | ITTL VPYTMDF |
| NO: 77 | | NO: 97 | VKG | NO: 117 | |
| SEQ ID | GYTFTGYWIE | SEQ ID | EILPGSGRSNSNEK | SEQ ID | GKLS |
| NO: 78 | | NO: 98 | FKG | NO: 118 | |
| SEQ ID | GYRFTDYPIH | SEQ ID | IISTYYGDVTNNPK | SEQ ID | NDLFAY |
| NO: 79 | | NO: 99 | FRG | NO: 119 | |
| SEQ ID | GYSITSDYAWN | SEQ ID | SYSGYTNYNPSLKS | SEQ ID | GDAYDVRRSTYYF |
| NO: 80 | | NO: 100 | | NO: 120 | DY |
| SEQ ID | LEEGYTFTVYWIE | SEQ ID | EILPGSGSTDYNEK | SEQ ID | GKLH |
| NO: 81 | | NO: 101 | FKG | NO: 121 | |
| SEQ ID | GFDFSKDWMS | SEQ ID | EINPDSRTINYAPSL | SEQ ID | WDYDGGMDY |
| NO: 82 | | NO: 102 | KD | NO: 122 | |
| SEQ ID | GFDFSRYWMS | SEQ ID | EINPDSSTINYTPSL | SEQ ID | PGYGNLFAY |
| NO: 83 | | NO: 103 | KD | NO: 123 | |
| SEQ ID | GYTFTSYVIH | SEQ ID | YIINPFSDGTKFTEK | SEQ ID | RGPYYYGTAMDY |
| NO: 84 | | NO: 104 | FKG | NO: 124 | |
| SEQ ID | GYTITIYWMQ | SEQ ID | AIYPGDGDTRYPQ | SEQ ID | GGSTNYDYDGFAY |
| NO: 85 | | NO: 105 | KFKG | NO: 125 | |
| SEQ ID | GYTFSNYWIE | SEQ ID | EILPGRGSTNYNEK | SEQ ID | GKQY |
| NO: 86 | | NO: 106 | FKG | NO: 126 | |
| | | | | SEQ ID | SALPRYDAMDY |
| | | | | NO: 127 | |
| | | | | SEQ ID | SGLPRYDGMDY |
| | | | | NO: 128 | |
| | | | | SEQ ID | SGLPHYDGMDY |
| | | | | NO: 129 | |

TABLE 4

| | Amino acid | | Amino acid | | Amino acid |
|---|---|---|---|---|---|
| SEQ ID | sequence of | SEQ ID | sequence of | SEQ ID | sequence of |
| NO | LCDR1 | NO | LCDR2 | NO | LCDR3 |
| SEQ ID | RASQGISSWLA | SEQ ID | AASSLQS | SEQ ID | QQYNSYPRT |
| NO: 131 | | NO: 151 | | NO: 171 | |
| SEQ ID | RASQGISSWLA | SEQ ID | AASSLOS | SEQ ID | QQYNSYPFT |
| NO: 132 | | NO: 152 | | NO: 172 | |
| SEQ ID | RASQSIGSSLH | SEQ ID | YASQSFS | SEQ ID | HQSSSLPIT |
| NO: 133 | | NO: 153 | | NO: 173 | |
| SEQ ID | RASQSVSSSYLA | SEQ ID | GASSRAT | SEQ ID | QQYGSSPMYT |
| NO: 134 | | NO: 154 | | NO: 174 | |
| SEQ ID | RASQGISSWLA | SEQ ID | AASSLOS | SEQ ID | QQYNSYPYT |
| NO: 135 | | NO: 155 | | NO: 175 | |
| SEQ ID | RASQSVSIYLA | SEQ ID | DASNRAT | SEQ ID | QQRSNWPPFT |
| NO: 136 | | NO: 156 | | NO: 176 | |
| SEQ ID | KASQDIYPYLN | SEQ ID | RTNRLLD | SEQ ID | LQYDEFPLT |
| NO: 137 | | NO: 157 | | NO: 177 | |
| SEQ ID | RASESVDSYGKSF | SEQ ID | RASNLES | SEQ ID | QQSNEDPWT |
| NO: 138 | MH | NO: 158 | | NO: 178 | |
| SEQ ID | ITNTDIDDDMN | SEQ ID | EGNGLRP | SEQ ID | LQSDNLPLT |
| NO: 139 | | NO: 159 | | NO: 179 | |
| SEQ ID | RASESVDSFGNSF | SEQ ID | AASNLGS | SEQ ID | QQSKEVPRT |
| NO: 140 | MH | NO: 160 | | NO: 180 | |
| SEQ ID | KASQNVGTNVV | SEQ ID | SASYRYS | SEQ ID | QHYNSFPYT |
| NO: 141 | | NO: 161 | | NO: 181 | |
| SEQ ID | KASDHINNWLA | SEQ ID | GATTLET | SEQ ID | QQYWSIPYT |
| NO: 142 | | NO: 162 | | NO: 182 | |
| SEQ ID | RASQSVSNNLH | SEQ ID | YASQSIS | SEQ ID | QQSYSWPRT |
| NO: 143 | | NO: 163 | | NO: 183 | |
| SEQ ID | KSSQSLLNGGNQQ | SEQ ID | GASTRES | SEQ ID | QNDHTYPYT |
| NO: 144 | NSLA | NO: 164 | | NO: 184 | |
| SEQ ID | RTSENIYSYLAWY | SEQ ID | NANTLAE | SEQ ID | QHHY GIPFT |
| NO: 145 | QQKQLA | NO: 165 | | NO: 185 | |
| SEQ ID | RASQTVDYNGMSY | SEQ ID | AASNLDS | SEQ ID | QQCIEDPLT |
| NO: 146 | MH | NO: 166 | | NO: 186 | |
| SEQ ID | RASENIYSNLA | SEQ ID | AATNLAD | SEQ ID | QHFWITPFT |
| NO: 147 | | NO: 167 | | NO: 187 | |
| SEQ ID | RASESVDNYGISFM | SEQ ID | AASNLGS | SEQ ID | QQSK |
| NO: 148 | H | NO: 168 | | NO: 188 | |

1. A conjugate of Formula (I): 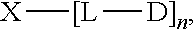
wherein:
n is a number from 1 to 20;
X is a polypeptide that binds PTK7 and comprises a heavy chain complementarity determining region 3 (HCDR3) comprising an amino acid sequence that has at least 85% homology to SEQ ID NO: 7;
L is a linker that covalently links X to D;
D is a drug, and
L-D has a structure of Formula (II): 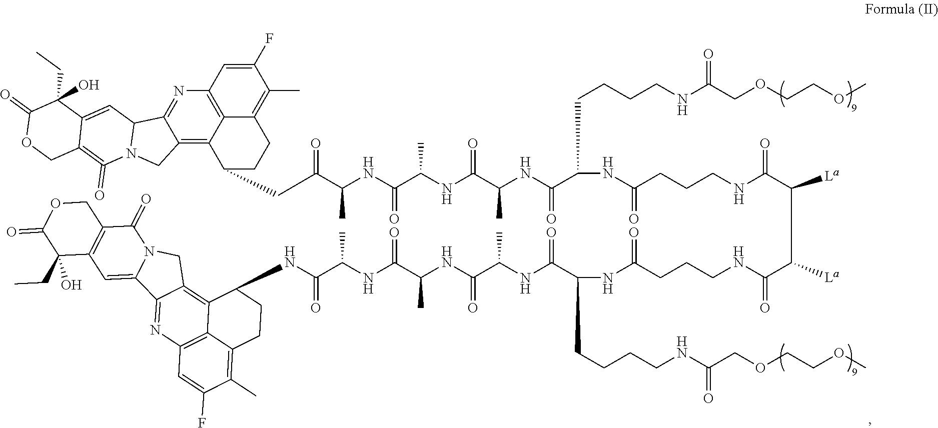
or a stereoisomer thereof, or a pharmaceutically acceptable salt thereof,
wherein each L^{a} is independently

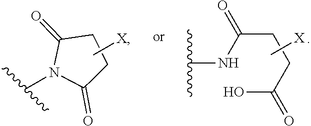

2. The conjugate of claim 1, wherein the polypeptide comprises an amino acid sequence with at least 75%, at least 80%, at least 85%, at least 90%, at least 95%, or at least 99% homology to SEQ ID NO: 1.

3. The conjugate of claim 1, wherein the polypeptide comprises an amino acid sequence with at least 75%, at least 80%, at least 85%, at least 90%, at least 95%, or at least 99% homology to SEQ ID NO: 2.

4. The conjugate of claim 1, wherein the polypeptide comprises a first amino acid sequence with at least 75%, at least 80%, at least 85%, at least 90%, at least 95%, or at least 99% homology to SEQ ID NO: 1; and a second amino acid sequence with at least 75%, at least 80%, at least 85%, at least 90%, at least 95%, or at least 99% homology to SEQ ID NO: 2.

5. The conjugate of claim 4, wherein the polypeptide comprises a first amino acid sequence that is SEQ ID NO: 1; and a second amino acid sequence that is SEQ ID NO: 2.

6. The conjugate of claim 1, wherein the polypeptide comprises an amino acid sequence that is at least 50 contiguous amino, at least 100 contiguous amino acids, at least 150 contiguous amino, or at least 200 contiguous amino acids homology to SEQ ID NO: 1.

7. The conjugate of claim 1, wherein the polypeptide comprises an amino acid sequence that is at least 50 contiguous amino, at least 100 contiguous amino acids, at least 150 contiguous amino, or at least 200 contiguous amino acids homology to SEQ ID NO: 2.

8. The conjugate of claim 1, wherein the polypeptide comprises an amino acid sequence with at least 75%, at least 80%, at least 85%, at least 90%, at least 95%, or at least 99% homology to SEQ ID NO: 3.

9. The conjugate of claim 1, wherein the polypeptide comprises an amino acid sequence with at least 75%, at least 80%, at least 85%, at least 90%, at least 95%, or at least 99% homology to SEQ ID NO: 4.

10. The conjugate of claim 1, wherein the polypeptide comprises a first amino acid sequence with at least 75%, at least 80%, at least 85%, at least 90%, at least 95%, or at least 99% homology to SEQ ID NO: 3; and a second amino acid sequence with at least 75%, at least 80%, at least 85%, at least 90%, at least 95%, or at least 99% homology to SEQ ID NO: 4.

11. The conjugate of claim 10, wherein the polypeptide comprises a first amino acid sequence that is SEQ ID NO: 3; and a second amino acid sequence that is SEQ ID NO: 4.

12. The conjugate of claim 1, wherein the polypeptide comprises an amino acid sequence that is at least 50 contiguous amino acids or at least 100 contiguous amino acids homology to SEQ ID NO: 3.

13. The conjugate of claim 1, wherein the polypeptide comprises an amino acid sequence that is at least 50 contiguous amino acids or at least 100 contiguous amino acids homology to SEQ ID NO: 4.

14. The conjugate of claim 1, wherein the polypeptide comprises a heavy chain complementarity determining region 1 (HCDR1) comprising an amino acid sequence with at least 75%, at least 80%, at least 85%, at least 90%, at least 95%, or at least 99% homology to SEQ ID NO: 5.

15. The conjugate of claim 1, wherein the polypeptide comprises a heavy chain complementarity determining region 2 (HCDR2) comprising an amino acid sequence with at least 75%, at least 80%, at least 85%, at least 90%, at least 95%, or at least 99% homology to SEQ ID NO: 6.

16. The conjugate of claim 1, wherein the polypeptide comprises a heavy chain complementarity determining region 3 (HCDR3) comprising an amino acid sequence that is at least 90%, at least 95%, or at least 99% homology to SEQ ID NO: 7.

17. The conjugate of claim 1, wherein the polypeptide comprises a light chain complementarity determining region 1 (LCDR1) comprising an amino acid sequence with at least 75%, at least 80%, at least 85%, at least 90%, at least 95%, or at least 99% homology to SEQ ID NO: 8.

18. The conjugate of claim 1, wherein the polypeptide comprises a light chain complementarity determining region 2 (LCDR2) comprising an amino acid sequence with at least 75%, at least 80%, at least 85%, at least 90%, at least 95%, or at least 99% homology to SEQ ID NO: 9.

19. The conjugate of claim 1, wherein the polypeptide comprises a light chain complementarity determining region 3 (LCDR3) comprising an amino acid with at least 75%, at least 80%, at least 85%, at least 90%, at least 95%, or at least 99% homology to SEQ ID NO: 10.

20. (canceled)

21. The conjugate of claim 1, wherein the polypeptide is chimeric or humanized.

22. The conjugate of claim 1, wherein the polypeptide comprises a Fc region.

23. The conjugate of claim 22, wherein the Fc region comprises a human IgG1 Fc region.

24. The conjugate of claim 23, wherein the Fc region comprises at least one modification.

25. The conjugate of claim 24, wherein the at least one modification comprises a
L234A (Kabat numbering) mutation;
L235A (Kabat numbering) mutation; or
both L234A (Kabat numbering) mutation and L235A (Kabat numbering) mutation.

26. (canceled)

27. A pharmaceutical composition comprising a conjugate of Formula (I): 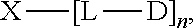
wherein:
n is a number from 1 to 20;
X is a polypeptide that binds PTK7 and comprises a heavy chain complementarity determining region 3 (HCDR3) comprising an amino acid sequence that has at least 85% homology to SEQ ID NO: 7;
L is a linker that covalently links X to D;
D is a drug, and
L-D has a structure of Formula (II): 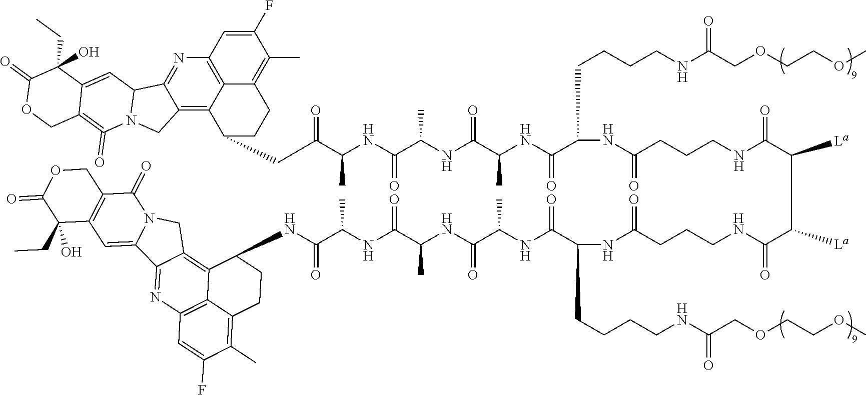
Formula (II),
or a stereoisomer thereof, or a pharmaceutically acceptable salt thereof,
wherein each L^{a} is independently 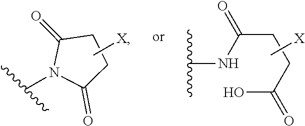
and a pharmaceutically acceptable diluent, carrier, or excipient.

28. (canceled)

29. A conjugate of Formula (I): 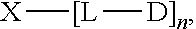
wherein:
n is a number from 1 to 20;
X is a polypeptide that binds to PTK7 and comprises an amino acid sequence that has at least 85% homology to SEQ ID NO: 7;
L is a linker that covalently links X to D; and
D is a drug.

30. (canceled)

31. The conjugate of claim 29, wherein X is a polypeptide that binds to an amino acid sequence that shares at least 70% homology to SEQ ID NO: 11.

32. The conjugate of claim 1, wherein n is 1-3.

33. The conjugate of claim 29, wherein n is 1-3.

34. A conjugate of Formula (I): 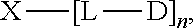
wherein:
n is 3;
X is a polypeptide that binds PTK7 and comprises:
a HCDR1 comprising an amino acid sequence that is SEQ ID NO: 5;
a HCDR2 comprising an amino acid sequence that is SEQ ID NO: 6;
a HCDR3 comprising an amino acid sequence that is SEQ ID NO: 7;
a LCDR1 comprising an amino acid sequence that is SEQ ID NO: 8;
a LCDR2 comprising an amino acid sequence that is SEQ ID NO: 9; and
a LCDR3 comprising an amino acid sequence that is SEQ ID NO: 10;
L is a linker that covalently links X to D;
D is a drug, and
L-D has a structure of Formula (II): 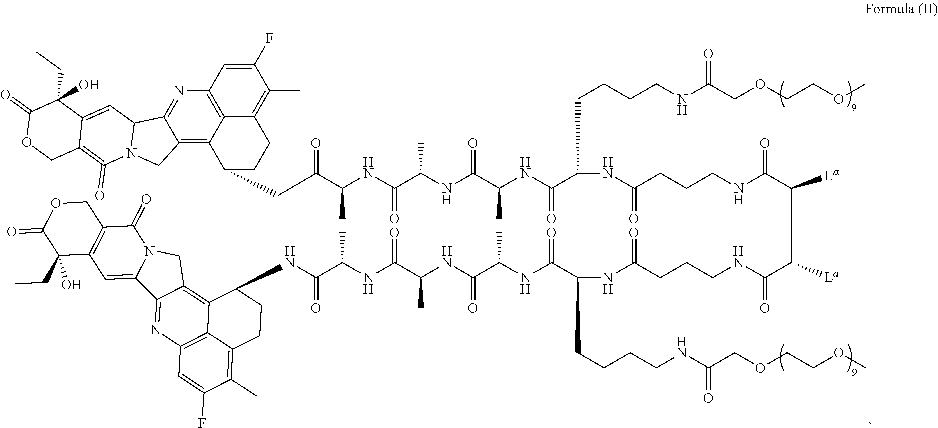
or a stereoisomer thereof, or a pharmaceutically acceptable salt thereof,
wherein each L^{a} is independently [chemical structure]


SEQUENCE LISTING (189 sequences)

SEQ ID NO: 1 - protein, 450 aa, synthetic construct
QVQLVQSGAEVKKPGSSVKVSCKASGYTFTDYVIHWVRQAPGQGLEWMGYINPWSEGTKYNEKFKGRVTLTSDKSTSTAYMELSSLRSEDTAVYYCSRSGLPRYDAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 218 aa, synthetic construct
DIVLTQSPASLAVSPGQRATITCRASQSVDYNALTYMNWLQQKPGQPPKLLIYAASTLESGVPARFSGSGSGTDFTLTINPVEAEDTANYYCQQSFRDPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 120 aa, synthetic construct
QVQLVQSGAEVKKPGSSVKVSCKASGYTFTDYVIHWVRQAPGQGLEWMGYINPWSEGTKYNEKFKGRVTLTSDKSTSTAYMELSSLRSEDTAVYYCSRSGLPRYDAMDYWGQGTTVTVSS

SEQ ID NO: 4 - protein, 111 aa, synthetic construct
DIVLTQSPASLAVSPGQRATITCRASQSVDYNALTYMNWLQQKPGQPPKLLIYAASTLESGVPARFSGSGSGTDFTLTINPVEAEDTANYYCQQSFRDPRTFGQGTKLEIK

SEQ ID NO: 5 - protein, 10 aa, synthetic construct
GYTFTDYVIH

SEQ ID NO: 6 - protein, 17 aa, synthetic construct
YINPWSEGTKYNEKFKG

SEQ ID NO: 7 - protein, 11 aa, synthetic construct
SGLPRYDAMDY

SEQ ID NO: 8 - protein, 15 aa, synthetic construct
RASQSVDYNALTYMN

SEQ ID NO: 9 - protein, 7 aa, synthetic construct
AASTLES

SEQ ID NO: 10 - protein, 9 aa, synthetic construct
QQSFRDPRT

SEQ ID NO: 11 - protein, 88 aa, synthetic construct
IEDMPLFEPRVFTAGSEERVTCLPPKGLPEPSVWWEHAGVRLPTHGRVYQKGHELVLANIAESDAGVYTCHAANLAGQRRQDVNITVA

SEQ ID NO: 12-70 - skipped (59 entries)

SEQ ID NO: 71 - protein, 10 aa, synthetic construct
GFIFSNYAMH

SEQ ID NO: 72 - protein, 10 aa, synthetic construct
GFTFSSYAFH

SEQ ID NO: 73 - protein, 10 aa, synthetic construct
GFTFSTYLMY

SEQ ID NO: 74 - protein, 10 aa, synthetic construct
GFTFSSYGMH

SEQ ID NO: 75 - protein, 12 aa, synthetic construct
GFSLSTSNMGVG

SEQ ID NO: 76 - protein, 10 aa, synthetic construct
GYTFTDYAVH

SEQ ID NO: 77 - protein, 10 aa, synthetic construct
GFDFSRYWMS

SEQ ID NO: 78 - protein, 10 aa, synthetic construct
GYTFTGYWIE

SEQ ID NO: 79 - protein, 10 aa, synthetic construct
GYRFTDYPIH

SEQ ID NO: 80 - protein, 11 aa, synthetic construct
GYSITSDYAWN

SEQ ID NO: 81 - protein, 13 aa, synthetic construct
LEEGYTFTVYWIE

SEQ ID NO: 82 - protein, 10 aa, synthetic construct
GFDFSKDWMS

SEQ ID NO: 83 - protein, 10 aa, synthetic construct
GFDFSRYWMS

SEQ ID NO: 84 - protein, 10 aa, synthetic construct
GYTFTSYVIH

SEQ ID NO: 85 - protein, 10 aa, synthetic construct
GYTITIYWMQ

SEQ ID NO: 86 - protein, 10 aa, synthetic construct
GYTFSNYWIE

SEQ ID NO: 87-90 - skipped (4 entries)

SEQ ID NO: 91 - protein, 17 aa, synthetic construct
VISYDGNNKYYADSVKG

SEQ ID NO: 92 - protein, 17 aa, synthetic construct
VISYDGSIKYYADSVKG

SEQ ID NO: 93 - protein, 16 aa, synthetic construct
AIGSGGDTYYADSVKG

SEQ ID NO: 94 - protein, 17 aa, synthetic construct
VIWDDGSNKYYVDSVKG

SEQ ID NO: 95 - protein, 16 aa, synthetic construct
HIWWDDDKYYSPSLKS

SEQ ID NO: 96 - protein, 17 aa, synthetic construct
VISTYNDYTYNNQDFKG

SEQ ID NO: 97 - protein, 17 aa, synthetic construct
DLNPDSSAINYVDSVKG

SEQ ID NO: 98 - protein, 17 aa, synthetic construct
EILPGSGRSNSNEKFKG

SEQ ID NO: 99 - protein, 17 aa, synthetic construct
IISTYYGDVTNNPKFRG

SEQ ID NO: 100 - protein, 14 aa, synthetic construct
SYSGYTNYNPSLKS

SEQ ID NO: 101 - protein, 17 aa, synthetic construct
EILPGSGSTDYNEKFKG

SEQ ID NO: 102 - protein, 17 aa, synthetic construct
EINPDSRTINYAPSLKD

SEQ ID NO: 103 - protein, 17 aa, synthetic construct
EINPDSSTINYTPSLKD

SEQ ID NO: 104 - protein, 18 aa, synthetic construct
YIINPFSDGTKFTEKFKG

SEQ ID NO: 105 - protein, 17 aa, synthetic construct
AIYPGDGDTRYPQKFKG

SEQ ID NO: 106 - protein, 17 aa, synthetic construct
EILPGRGSTNYNEKFKG

SEQ ID NO: 107-110 - skipped (4 entries)

SEQ ID NO: 111 - protein, 7 aa, synthetic construct
EVWSIDN

SEQ ID NO: 112 - protein, 6 aa, synthetic construct
TYYFDY

SEQ ID NO: 113 - protein, 4 aa, synthetic construct
GLGY

SEQ ID NO: 114 - protein, 17 aa, synthetic construct
DDYYGSGSFNSYYGTDV

SEQ ID NO: 115 - protein, 10 aa, synthetic construct
SNYGYAWFAY

SEQ ID NO: 116 - protein, 10 aa, synthetic construct
GNSYFYALDY

SEQ ID NO: 117 - protein, 11 aa, synthetic construct
ITTLVPYTMDF

SEQ ID NO: 118 - protein, 4 aa, synthetic construct
GKLS

SEQ ID NO: 119 - protein, 6 aa, synthetic construct
NDLFAY

SEQ ID NO: 120 - protein, 15 aa, synthetic construct
GDAYDVRRSTYYFDY

SEQ ID NO: 121 - protein, 4 aa, synthetic construct
GKLH

SEQ ID NO: 122 - protein, 9 aa, synthetic construct
WDYDGGMDY

SEQ ID NO: 123 - protein, 9 aa, synthetic construct
PGYGNLFAY

SEQ ID NO: 124 - protein, 12 aa, synthetic construct
RGPYYYGTAMDY

SEQ ID NO: 125 - protein, 13 aa, synthetic construct
GGSTNYDYDGFAY

SEQ ID NO: 126 - protein, 4 aa, synthetic construct
GKQY

SEQ ID NO: 127 - protein, 11 aa, synthetic construct
SALPRYDAMDY

SEQ ID NO: 128 - protein, 11 aa, synthetic construct
SGLPRYDGMDY

SEQ ID NO: 129 - protein, 11 aa, synthetic construct
SGLPHYDGMDY

SEQ ID NO: 130 - skipped

SEQ ID NO: 131 - protein, 11 aa, synthetic construct
RASQGISSWLA

SEQ ID NO: 132 - protein, 11 aa, synthetic construct
RASQGISSWLA

SEQ ID NO: 133 - protein, 11 aa, synthetic construct
RASQSIGSSLH

SEQ ID NO: 134 - protein, 12 aa, synthetic construct
RASQSVSSSYLA

SEQ ID NO: 135 - protein, 11 aa, synthetic construct
RASQGISSWLA

SEQ ID NO: 136 - protein, 11 aa, synthetic construct
RASQSVSIYLA

SEQ ID NO: 137 - protein, 11 aa, synthetic construct
KASQDIYPYLN

SEQ ID NO: 138 - protein, 15 aa, synthetic construct
RASESVDSYGKSFMH

SEQ ID NO: 139 - protein, 11 aa, synthetic construct
ITNTDIDDDMN

SEQ ID NO: 140 - protein, 15 aa, synthetic construct
RASESVDSFGNSFMH

SEQ ID NO: 141 - protein, 11 aa, synthetic construct
KASQNVGTNVV

SEQ ID NO: 142 - protein, 11 aa, synthetic construct
KASDHINNWLA

SEQ ID NO: 143 - protein, 11 aa, synthetic construct
RASQSVSNNLH

SEQ ID NO: 144 - protein, 17 aa, synthetic construct
KSSQSLLNGGNQQNSLA

SEQ ID NO: 145 - protein, 19 aa, synthetic construct
RTSENIYSYLAWYQQKQLA

SEQ ID NO: 146 - protein, 15 aa, synthetic construct
RASQTVDYNGMSYMH

SEQ ID NO: 147 - protein, 11 aa, synthetic construct
RASENIYSNLA

SEQ ID NO: 148 - protein, 15 aa, synthetic construct
RASESVDNYGISFMH

SEQ ID NO: 149-150 - skipped (2 entries)

SEQ ID NO: 151 - protein, 7 aa, synthetic construct
AASSLQS

SEQ ID NO: 152 - protein, 7 aa, synthetic construct
AASSLQS

SEQ ID NO: 153 - protein, 7 aa, synthetic construct
YASQSFS

SEQ ID NO: 154 - protein, 7 aa, synthetic construct
GASSRAT

SEQ ID NO: 155 - protein, 7 aa, synthetic construct
AASSLQS

SEQ ID NO: 156 - protein, 7 aa, synthetic construct
DASNRAT

SEQ ID NO: 157 - protein, 7 aa, synthetic construct
RTNRLLD

SEQ ID NO: 158 - protein, 7 aa, synthetic construct
RASNLES

SEQ ID NO: 159 - protein, 7 aa, synthetic construct
EGNGLRP

SEQ ID NO: 160 - protein, 7 aa, synthetic construct
AASNLGS

SEQ ID NO: 161 - protein, 7 aa, synthetic construct
SASYRYS

SEQ ID NO: 162 - protein, 7 aa, synthetic construct
GATTLET

SEQ ID NO: 163 - protein, 7 aa, synthetic construct
YASQSIS

SEQ ID NO: 164 - protein, 7 aa, synthetic construct
GASTRES

SEQ ID NO: 165 - protein, 7 aa, synthetic construct
NANTLAE

SEQ ID NO: 166 - protein, 7 aa, synthetic construct
AASNLDS

SEQ ID NO: 167 - protein, 7 aa, synthetic construct
AATNLAD

SEQ ID NO: 168 - protein, 7 aa, synthetic construct
AASNLGS

SEQ ID NO: 169-170 - skipped (2 entries)

SEQ ID NO: 171 - protein, 9 aa, synthetic construct
QQYNSYPRT

SEQ ID NO: 172 - protein, 9 aa, synthetic construct
QQYNSYPFT

SEQ ID NO: 173 - protein, 9 aa, synthetic construct
HQSSSLPIT

SEQ ID NO: 174 - protein, 10 aa, synthetic construct
QQYGSSPMYT

SEQ ID NO: 175 - protein, 9 aa, synthetic construct
QQYNSYPYT

SEQ ID NO: 176 - protein, 10 aa, synthetic construct
QQRSNWPPFT

SEQ ID NO: 177 - protein, 9 aa, synthetic construct
LQYDEFPLT

SEQ ID NO: 178 - protein, 9 aa, synthetic construct
QQSNEDPWT

SEQ ID NO: 179 - protein, 9 aa, synthetic construct
LQSDNLPLT

SEQ ID NO: 180 - protein, 9 aa, synthetic construct
QQSKEVPRT

SEQ ID NO: 181 - protein, 9 aa, synthetic construct
QHYNSFPYT

SEQ ID NO: 182 - protein, 9 aa, synthetic construct
QQYWSIPYT

SEQ ID NO: 183 - protein, 9 aa, synthetic construct
QQSYSWPRT

SEQ ID NO: 184 - protein, 9 aa, synthetic construct
QNDHTYPYT

SEQ ID NO: 185 - protein, 9 aa, synthetic construct
QHHYGIPFT

SEQ ID NO: 186 - protein, 9 aa, synthetic construct
QQCIEDPLT

SEQ ID NO: 187 - protein, 9 aa, synthetic construct
QHFWITPFT

SEQ ID NO: 188 - protein, 4 aa, synthetic construct
QQSK

SEQ ID NO: 189 - protein, 4 aa, synthetic construct
GGFG